(19) United States
(12) Patent Application Publication
(10) Pub. No.: US 2020/0129645 A1
(43) Pub. Date: Apr. 30, 2020

(54) CONTRAST AGENTS FOR MAGNETIC RESONANCE IMAGING

(71) Applicant: GEORG-AUGUST-UNIVERSITAET GOETTINGEN STIFTUNG OEFFENTLICHEN RECHTS, Goeltingen (DE)

(72) Inventors: Franc MEYER, Goettingen (DE); Jeremiah SCEPANIAK, Philadelphia, PA (US)

(73) Assignee: GEORG-AUGUST-UNIVERSITAET GOETTINGEN STIFTUNG OEFFENTLICHEN RECHTS, Goettingen (DE)

(21) Appl. No.: 16/624,125

(22) PCT Filed: Jun. 18, 2018
(86) PCT No.: PCT/EP2018/066104

§ 371 Date: Dec. 18, 2019

(30) Foreign Application Priority Data
Jun. 19, 2017 (GB) 1709715.5

(51) Int. Cl. A61K 49/10; A61B 5/055; A61K 49/00; C07D 213/04; C07D 231/10; C07D 233/04; C07D 233/54; C07D 241/06; C07D 239/06

(52) U.S. Cl. CPC A61K 49/103; A61B 5/055; A61K 49/0002; C07D 213/04; C07D 239/06; C07D 233/04; C07D 233/54; C07D 241/06; C07D 231/10

(57) ABSTRACT

The present invention provides contrast agents of the formula [N(A₁,A₂,A₃) M](counter ion(s)) for use in a diagnostic method practiced on the human or animal body. It also refers to the contrast agents, as well as pharmaceutical compositions containing same. Further, it relates to a method of in vitro medical imaging, especially of diagnostic imaging, comprising administering said compound to a sample.

This invention is relevant to the field of biological imaging, in particular Magnetic Resonance Imaging (MRI) in the clinical and veterinary setting. The invention derives from inorganic coordination chemistry, and is the first use of tripodal Schiff base ligands as defined in the claims to support first-row transition metals to provide water-soluble coordination complexes that act as paramagnetic chemical exchange saturation transfer (paraCEST) agents that will provide signal contrast in MRI.

The present invention provides contrast agents of the formula [N(A₁,A₂,A₃) M](counter ion(s)) for use in a diagnostic method practiced on the human or animal body as defined in the claims. It also refers to the contrast agents as defined in the claims, as well as pharmaceutical compositions containing same. Further, it relates to a method of in vitro medical imaging, especially of diagnostic imaging, comprising administering a compound as defined in the claims to a sample.

### BACKGROUND PRIOR ART

State of the Art: Metal-Containing MRI Contrast Agents
Acta Crystallographica 2004, C60, m177-m179, Hardie et al.; “{Tris[4-(1H-pyrazol-3-yl)-3-azabut-3-enyl]amine}iron(II) diperchlorate monohydrate” discloses ligands, however not in the context of MRI.
Brewer et al., Dalton Transactions March 2006, 5617-5629; “A DFT computational study of spin crossover in iron (III) and iron (II) tripodal imidazole complexes. A comparison of . . . ” discloses ligands, however not in the context of MRI.
Brewer et al., Dalton Transactions February 2006; 28(8), 1009-1019;
“Synthesis and characterization of manganese(II) and iron(III) d⁵ tripodal imidazole complexes. Effect of oxidation state, protonation state and ligand conformation on coordination number and spin state” discloses ligands, however not in the context of MRI.
Brewer et al., Inorg Chem. 2004, Apr. 5; 43(7), 2402-2415 “Proton Control of Oxidation and Spin State in a Series of Iron Tripodal Imidazole Complexes” discloses ligands, however not in the context of MRI.
U.S. Pat. No. 5,820,851A of Hoechst discloses pyridine-based ligands, however not in the context of MRI.
U.S. Pat. No. 5,869,026 of Hoechst discloses carboxamide-based ligands for use in the context of contrast agents.
WO 2006/114739A2 of Philips discloses how to perform MRI,
WO2009/072079A2 of Philips discloses polymer-supported macrocyclic paraCEST contrast agents.
WO 2009/126289A2 of Beth Israel Deaconess Medical Center, Inc. discloses how to perform MRI.
WO 2012/155076A2 of Sarina discloses macrocyclic MRT contrast agents.
WO 2012/155085A1 of Sarina discloses substituted pyridine-based ligands.
JP2000119247 discloses the preparation of specific transition metal complex catalysts, but not MRI contrast agents.
JPH01272568 discloses the preparation of specific tris[2-(2-pyridylmethyl)aminoethyl]amine-iron complexes as oxygen scavenging compounds, but not as MRI contrast agents.

Inorganic coordination chemistry has provided clinically utilized MRI contrast agents in the form of lanthanides supported by macrocyclic ligands, chelates, and iron oxide nanoparticles. These agents function through modification of either the longitudinal (T₁) or transverse (T₂) relaxation time of water protons in the local environment. Contrast agents accumulate in the extracellular space of lesions and regions of increased vasculature, reporting on concentration (accumulation) rather than local tissue conditions. Traditional contrast agents do not detect changes associated with cancer progression, such as elevated temperature or the acidic extracellular pH of tumors. Chemical exchange saturation transfer (CEST) is another avenue to produce contrast in MRI; the technique relies upon proton exchange between a contrast agent and bulk water and may inherently report on local tissue conditions such as pH or temperature A subfield of CEST, paramagnetic chemical exchange saturation transfer (paraCEST) is an underexplored avenue towards obtaining contrast in MRI. ParaCEST compounds contain paramagnetically shifted labile —OH, —NH, or metal-bound —H₂O protons that undergo exchange with the protons of bulk water. ParaCEST agents have not yet been approved for clinical use.

State of the Art: ParaCEST Contrast Agents

The primary requirement for paraCEST contrast to be realized is a hyperfine chemical shift difference between the exchangeable proton and bulk water (Δω) greater than the exchange rate constant (kₑₓ). Hyperfine proton chemical shifts arise through contact and pseudo-contact interactions between the nuclear spin of a proton and the unpaired electrons of a metal ion. The metal-ligand interactions are primarily electrostatic in the Ln^{III} series due to shielding of the 4f orbitals, in which hyperfine chemical shifts arise primarily through pseudo-contact interactions. Transition metal complexes, on the other hand, exhibit greater covalency in the metal-ligand bonds, potentially providing greater contributions to proton hyperfine shifts from contact interactions through multiple chemical bonds.

Large values of Δω have three advantages for paraCEST agents. First, a large Δω imparts tolerance towards an increased kₑₓ for labile protons, allowing fast exchanging hydroxyl or amine protons to be utilized in addition to slower exchanging amide protons, contributing to more exchange occurrences and providing greater contrast enhancement. The second advantage of a large Δω is the prevention of inadvertent saturation of bulk water from the presaturation pulse, which limits attainment of contrast. Lastly, endogenous macromolecules with labile protons contribute to background interference through magnetization transfer, which is more pronounced closer to the resonance frequency of H₂O, becoming much less intense at frequencies >50 kHz. Utilization of paraCEST agents with large Δω values minimizes magnetization transfer from exchangeable protons of endogenous macromolecules during the presaturation pulse, allowing for a reduction of “noise” resulting from magnetization transfer.

The development of paraCEST agents has focused on the lanthanides (Ln^{III}), particularly Eu^{III} supported by the appended Cyclen tetraazamacrocycle. Lanthanide complexes supported by amide-appended DOTAM and derivatives (e.g. DTMA among others) as well as the alcohol-appended S-THP ligand have also been studied. These compounds exhibit a great deal of versatility, exhibiting properties that allow for the sensing of temperature, pH, metabolites, metal ions, as well as proteins and enzymes.

Paramagnetic first row transition metal complexes, particularly high spin (HS) Fe^{II}, Co^{II}, and Ni^{II}, offer potentially biotolerated alternatives to Ln^{III}-based paraCEST contrast agents, as a biological mechanism for the regulation of trivalent lanthanides (Ln^{III}) is unknown. Utilization of macrocyclic ligands to support Fe^{II}, Co^{II}, and Ni^{II} has already been demonstrated, and has provided a number of complexes that exhibit paraCEST effects of 13% to 39% at 10 mM and 37° C., though formation of free macrocycle arising from complex dissociation and the disruption of the action of Ca^{II} in vivo is still a health concern. The development of first-row transition metal paraCEST contrast agents ligated by non-macrocyclic ligands has been met with less attention. The handful of known examples include a ferromagnetically coupled Cu′ dimer that provides a CEST effect of 14% at 10 mM and 37° C., and an Fe^{II} complex ligated by two dipyrazolylpyridine ligands that provides a 17% CEST effect at 10 mM and 25° C. The aforementioned contribution by Jeon et al. discloses pyridine contrast agents, which are build up with a different structure of the linker/column between the pyridine and the “key” N at the center of the AN1N2N3 structure as compared with the compounds disclosed herein.

Technical Deficiencies of Known MRI Contrast Agents

Magnetic resonance imaging (MRI) provides 3-D images of soft tissue deep in the body utilizing non-ionizing radio frequency radiation where detection of abundant water protons allows anatomical features to be visualized. Image contrast enhancement is provided in 40-50% of MRI scans through administration of a contrast agent to further delineate regions of interest. Image contrast enhancement requires the collection of a baseline scan and a contrast enhanced scan to compile a composite image. Known contrast agents have a number of deficiencies.

1) Contrast agents currently in clinical use, whether a T₁ or T₂ agent, are always “on” requiring administration of a contrast agent after collection of a baseline scan; making contrast enhancement a time consuming process that increases the cost of the imaging modality. Conversely, paraCEST agents allow for the contrast agent to be turned “on and off” selectively. In principle this behavior allows collection of a contrast enhanced scan prior to collecting a baseline, or alternatively administering the contrast agent during the baseline scan, in both cases to provide a composite image in a shorter time period with concurrent cost-savings. 2) Clinically utilized contrast agents primarily rely upon Gd^{III} supported by macrocyclic ligands, making metal-ligand dissociation a two-fold health hazard. A biological mechanism for the regulation of trivalent lanthanides (Ln^{III}) is unknown, making accumulation of free Ln^{III} harmful, especially for patients with compromised kidney function. Free macrocycle arising from complex dissociation may also be harmful as it can bind to Ca^{II} and disrupt the normal action of calcium in vivo. Clinically utilized T₁ Ln^{III} agents as well as most paraCEST contrast agents developed to date are based on Ln^{III} metal ions supported by macrocyclic ligands; or Fe^{II}, Co^{II} and Ni^{II} metal ions supported by macrocycles. The potential biotoxicity resulting from complex dissociation of these complexes to give free macrocyclic ligand that can in-turn interfere with the action of Ca^{II} in vivo is thus a concern for transition metal paraCEST complexes supported by macrocyclic ligands, as well as Ln^{III} based paraCEST and T₁ agents.

3) The reliance on trivalent lanthanides to provide coordination complexes that facilitate T₁ relaxation or paraCEST, is also a concern due to the fluctuating cost and availability of lanthanides, primarily produced from Asian sources. The relatively high price of lanthanides, and the volatility in their price is a result of an increased use of lanthanides in emerging technologies, the environmentally costly extraction and purification processes to obtain these elements, and the forces of political whimsy and protectionist trade policies.

Previous Attempts is to Remedy Technical Deficiencies of MRI Contrast Agents

The technical deficiencies associated with T₁ and T₂ contrast agents have to an extent been remedied by the development of Ln^{III} based paraCEST contrast agents. In turn paraCEST contrast agents composed of first row transition metal ions supported by macrocyclic ligands have been developed to provide alternatives to lanthanide based paraCEST contrast agents. Use of transition metals in place of lanthanides has two advantages: 1) Use of transition metals avoids toxicity issues arising from the use of Ln^{III} based agents and 2) Substituting economically and environmentally costly lanthanides with more abundant transition metals will have financial and environmental benefits. The use of macrocyclic ligands to support transition metal ions in place of lanthanides has provided effective paraCEST contrast agents. Unfortunately, metal-ligand dissociation to provide free macrocycle is still a concern, as these macrocycles can bind and interfere with the action of Ca^{II} in vivo.

Underlying Task the Invention May be a Solution for

The development of lanthanide based paraCEST contrast agents, and their first-row transition metal counterparts has provided a series of water soluble complexes stable towards aerobic oxidation that exhibit a paramagnetic CEST effect. Unfortunately few efforts have been made towards the development of transition metal based paraCEST agents that are supported by non-macrocyclic ligands in order to remedy the problems associated with formation of free macrocycle in solution upon complex dissociation.

Though some paraCEST contrast agents are available, they suffer from deficiencies, and it is an object of the present invention to provide contrast agents that overcome at least one of the above problems.

### SUMMARY OF THE INVENTION

The present invention expands the breadth of known transition metal-based paraCEST agents beyond the few examples of Fe^{II}, Co^{II}, and Ni^{II} that are supported by macrocyclic ligands. So far, only one contribution has explored a non-macrocyclic Fe^{II} compound exhibiting paraCEST properties. This compound has a different structure as compared with the compounds of the present invention. Because the scientific community has an insufficient understanding as to how paramagnetism on a metal center alters the chemical shift of protons on the coordinating ligand predictions regarding the suitability of a paramagnetic metal ion in a particular ligand framework cannot be made. While reports of Fe^{II} perchlorate complexes of ligands A₁, A₂, and A₃ as described herein had reported paramagnetism, description of the solution-state structure as determined by ¹H NMR were absent in most cases, and no report demonstrated solubility and stability in water, a prerequisite for paraCEST efficacy. It was therefore unexpectedly found that the core structures of ligands A₁, A₂ and A₃ as described herein provide highly beneficial properties. Since ligands A₁, A₂ and A₃ possess different nitrogen-containing coordination arms (imidazole or pyrazole) but are built upon a common tris-azabutylamine foundation it is rational to conclude that ligands 4-7, built upon the same tris-azabutylamine foundation with analogous nitrogen-containing coordination arms will exhibit paraCEST efficacy as well.

A second, non-macrocyclic bimetallic ferromagnetically coupled Cu complex has also recently demonstrated paraCEST efficacy (Du, K.; Harris, T. D. J. Am. Chem. Soc. 2016, 138 (25), 7804-7807). The paraCEST complexes described herein utilize tripodal Schiff base ligands to support divalent Fe and Co in a pseudo-octahedral coordination environment. These complexes are stable for upwards of 7 days under anaerobic conditions in aqueous solution buffered to physiological pH and ionic strength with no appreciable decrease in paraCEST efficacy. These complexes exhibit paraCEST efficacy at 37° C. ranging from 10% for [L^{3H3}Fe]²⁺(OTf)₂, 11% for [L^{1H3}Fe]²⁺(Cl)₂, 14% for [L^{2H3}Fe]²⁺(OTf)₂, 33% for [L^{1H3}Fe]²⁺(OTf)₂, and 48% for [L^{1H3}Co]₂₊Cl₂, when a 2 second presaturation pulse at a power of 18.7 μT is used; thus providing paraCEST complexes that meet or exceed the efficacy of previously reported first-row transition metal paraCEST agents supported by macrocyclic ligands.

The practical use of the coordination complexes disclosed herein as paraCEST contrast agents for MRI will require administration of contrast agent intravenously as a saline solution buffered to a physiologically relevant pH and ionic strength (pH 7.0 and 100 mM NaCl). Rationally, the contrast agents would be prepared in saline solutions under anaerobic conditions to extend shelf-life, and stored at lowered temperatures to extend complex stability in solution. Administration of the contrast agent orally cannot be ruled-out as these complexes are anticipated to be stabilized in mildly acidic environments, though the strongly acidic environment of the human stomach may induce decomposition of the metal complexes.

Due to the fundamental nature of the paraCEST experiment, the contrast agent may be administered prior-to or during the baseline scan that is collected to provide a contrast enhanced composite magnetic resonance image. This can be accomplished for paraCEST contrast agents, but not T₁ or T₂ contrast agents, because as mentioned previously, paraCEST contrast agents require the use of a presaturation pulse at a specific frequency, enabling these contrast agents to be turned on and off. Similarly, due to the nature of the paraCEST contrast experiment, a contrast enhanced scan can in principle be collected prior to the baseline scan, as the contrast agent can be turned “on and off” selectively.

In the context of the present invention, is has unexpectedly been found that the compounds as defined in the claims exhibit kinetic stability under aerobic conditions in the dissolved state at physiologically relevant pH and ionic strength. The compounds are stable at increased temperatures and different pH-values. But even more importantly, the compounds show high paraCEST efficacy. Surprisingly, Z-spectra collected at the acidic pH of diseased tissue (pH 6.8) provide greater contrast (FIG. 21-26) as compared with neutral or slightly alkaline pH of healthy tissue (pH 7.0, 7.4), thus providing paraCEST agents that are “activated” by the acidic pH associated with diseased tissue The compounds may thus serve for clinical use.

The invention thus refers to a contrast agent of the formula [N(A₁,A₂,A₃) M](counter ion(s)) as defined in the claims for use in a diagnostic method practiced on the human or animal body. It further refers to a contrast agent as defined in the claims as well as a pharmaceutical composition comprising said contrast agent and at least one pharmaceutically acceptable excipient. It also relates to a method of in vitro medical imaging, especially of diagnostic imaging, comprising administering a compound as defined in the claims to a sample.

### DESCRIPTION, OF THE FIGURES

FIG. 1. Tripodal Schiff Base ligands disclosed herein.

FIG. 2. ¹H NMR spectra [L^{1H3}Fe](OTf)₂ in CD₃CN under anaerobic and anhydrous conditions at 25° C.

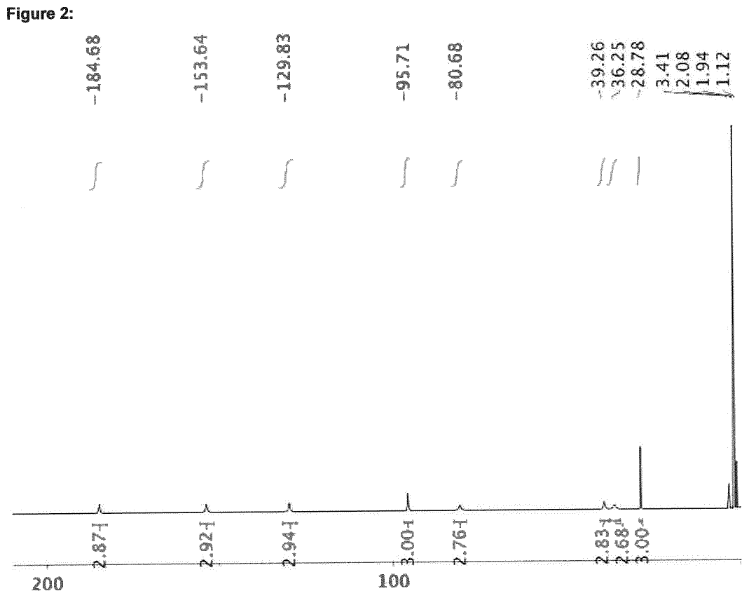

FIG. 3. ¹⁹F NMR spectra of [L^{1H3}Fe](OTf)₂ in CD₃CN under anaerobic and anhydrous conditions at 25° C.

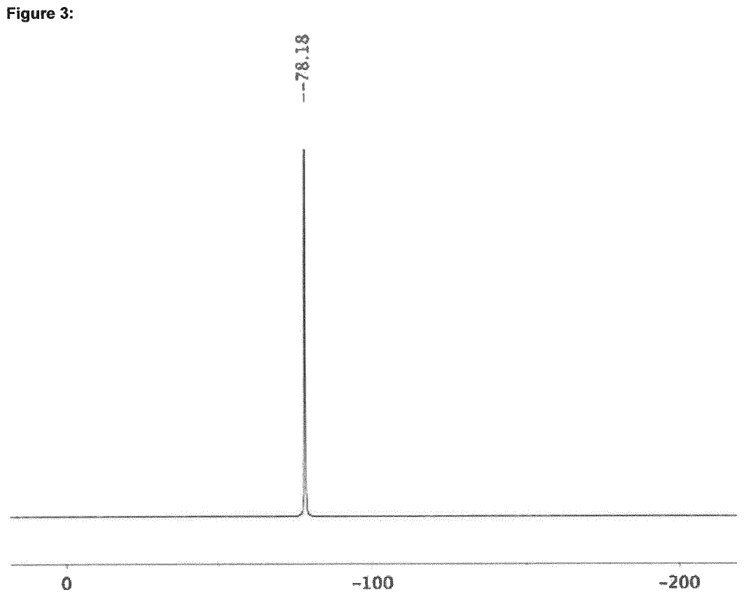

FIG. 4. ¹H NMR spectra of [L^{1H3}Fe](OTf)₂ in CD₃OD under anaerobic and anhydrous conditions at 25° C.

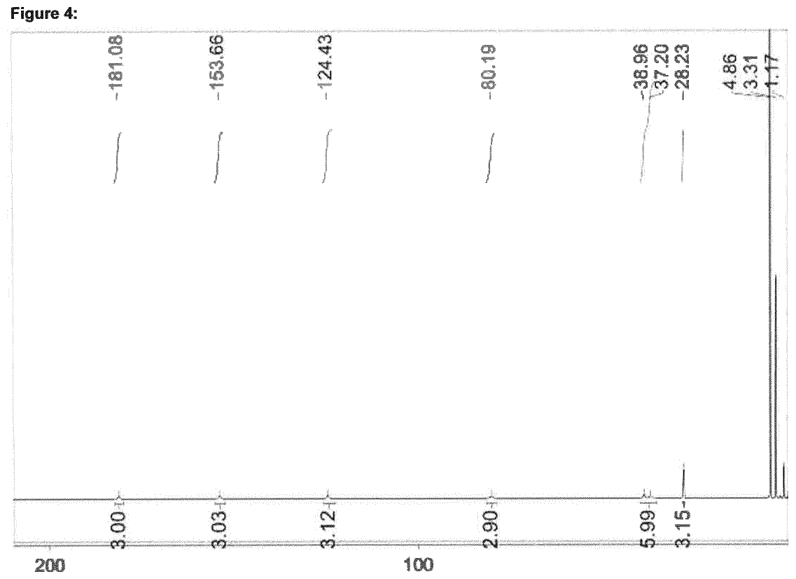

FIG. 5. ¹⁹F NMR spectra of [L^{1H3}Fe](OTf)₂ in CD₃OD under anaerobic and anhydrous conditions at 25° C.

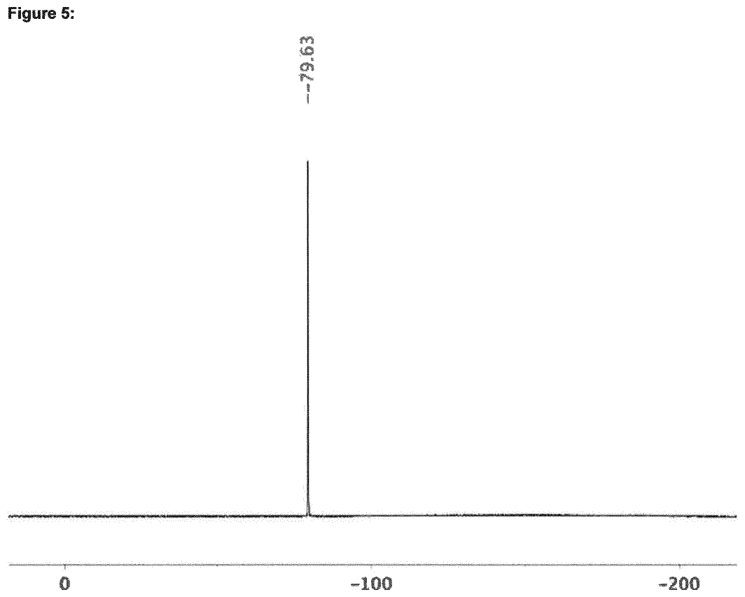

FIG. 6. ¹H NMR spectra of [L^{1H3}Fe](Cl)₂ in CD₃OD under anaerobic and anhydrous conditions at 25° C.

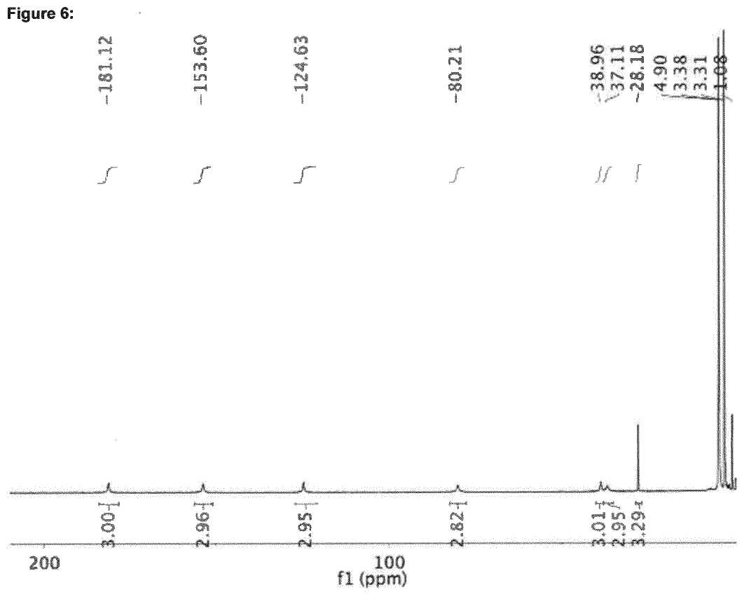

FIG. 7. ¹H NMR spectra of [L^{1H3}Co](Cl)₂ in CD₃OD under anaerobic and anhydrous conditions at 25° C.

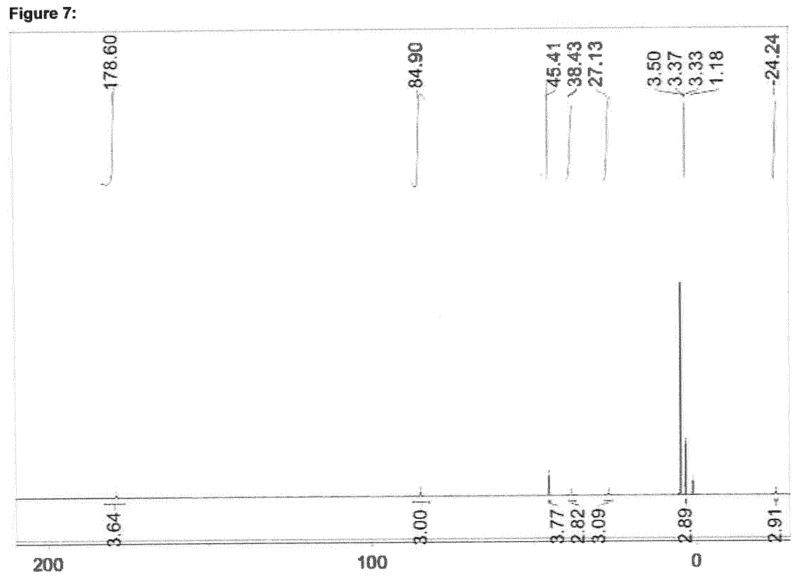

FIG. 8. ¹H NMR spectra of [L^{2H3}Fe](OTf)₂ in CD₃CN under anaerobic and anhydrous conditions.

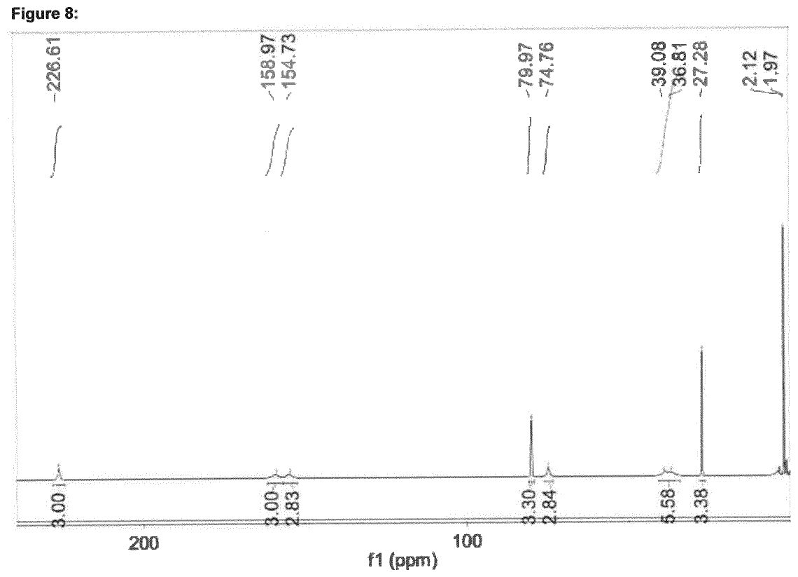

FIG. 9. ¹⁹F NMR spectra of [L^{2H3}Fe](OTf)₂ in CD₃CN under anaerobic and anhydrous conditions at 25° C.

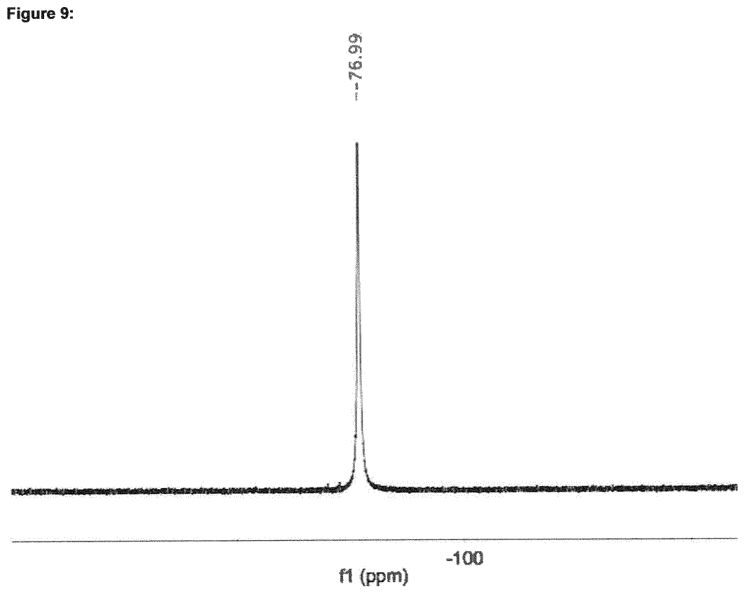

FIG. 10. ¹H NMR spectra of [L^{2H3}Fe](OTf)₂ in CD₃OD under anaerobic and anhydrous conditions.

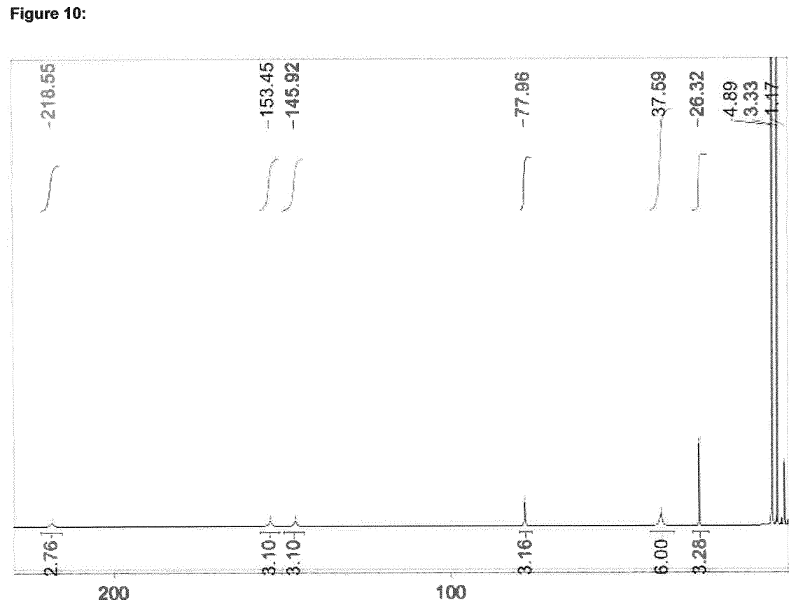

FIG. 11. ¹⁹F NMR spectra of [L^{2H3}Fe](OTf)₂ in CD₃OD under anaerobic and anhydrous conditions.

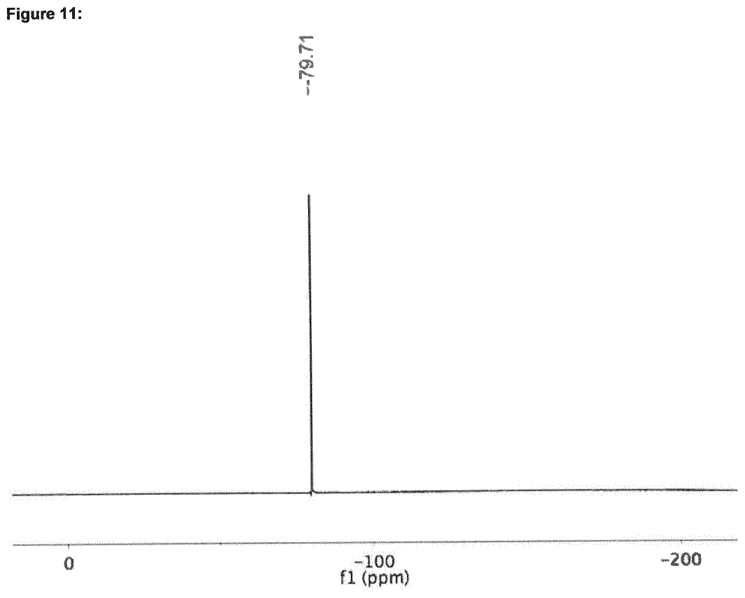

FIG. 12. ¹H NMR spectra of [L^{3H3}Fe](OTf)₂ in CD₃CN under anaerobic and anhydrous conditions.

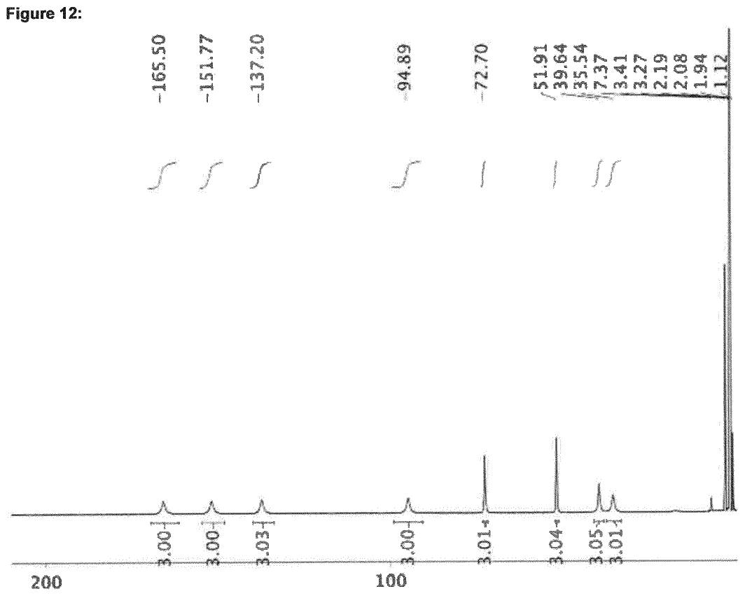

FIG. 13. ¹⁹F NMR spectra of [L^{3H3}Fe](OTf)₂ in CD₃CN under anaerobic and anhydrous conditions at 25° C.

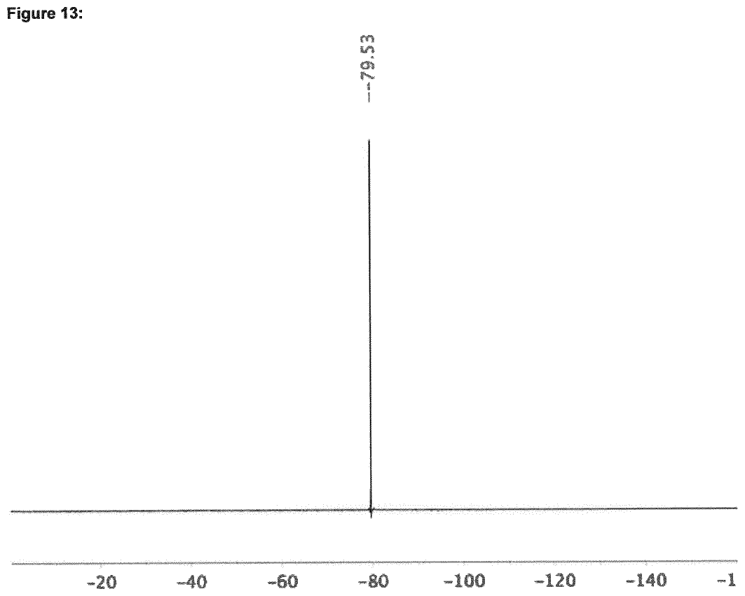

FIG. 14. ¹H NMR spectra of [L^{3H3}Fe](OTf)₂ in CD₃OD under anaerobic and anhydrous conditions.

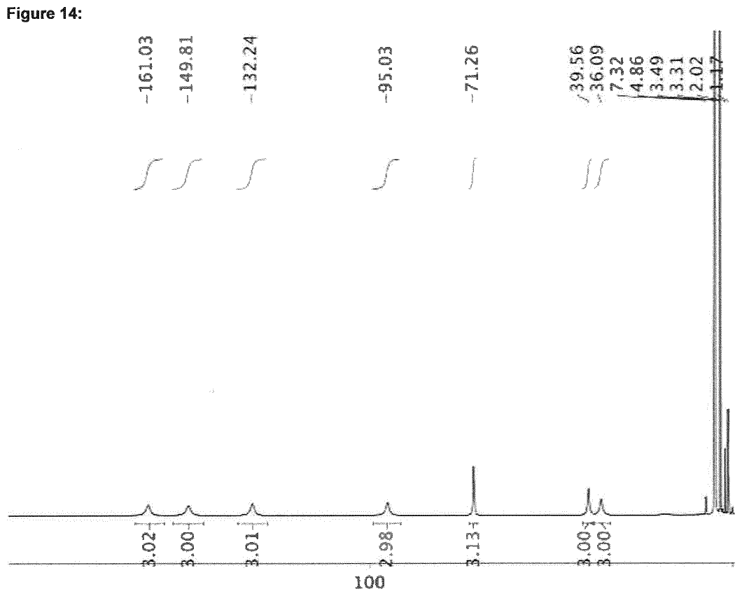

FIG. 15. ¹⁹F NMR spectra of [L^{3H3}Fe](OTf)₂ in CD₃OD under anaerobic and anhydrous conditions.

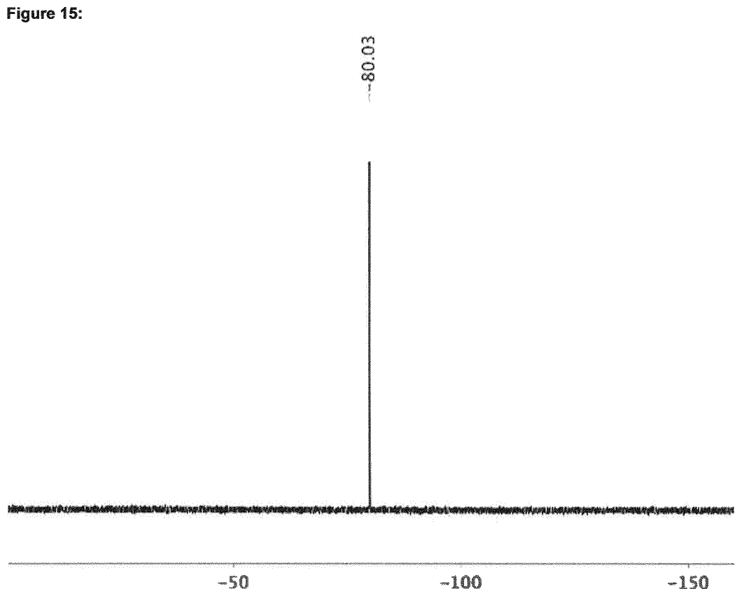

FIG. 16. ¹H NMR spectra of free ligand L^{1H3} in D₂O. It should be noted that the resonance at δ 9.69 is not observed in the paramagnetic spectra of freshly prepared D₂O solutions of [L^{1H3}Fe](OTf)₂, [L^{1H3}Fe](Cl)₂, or [L^{1H3}Co](Cl)₂, thus providing a spectroscopic fingerprint to monitor complex stability in D₂O over time.

FIG. 17. ¹H NMR spectra [L^{1H3}Fe](OTf)₂ in D₂O under anaerobic conditions at 25 C at 0 hr. Paramagnetic peaks are integrated against an internal capillary containing C₆D₆/C₆H₆ (δ 6.81) (80:20).

FIG. 18. ¹H NMR spectra [L^{1H3}Fe](OTf)₂ in D₂O under anaerobic conditions at 25 C after 13 days. Paramagnetic peaks are integrated against an internal capillary containing C₆D₆/C₆H₆ (δ 6.73), (80:20). Comparing the 0 hr/h spectra against the spectra collected after 13 days indicates less than 5% decomposition of the paramagnetic signals when integrated against the internal standard, while a analysis of the integration of the “fingerprint” resonance for L^{1H3} at 9.67 shows formation of approximately 1% free ligand in solution.

FIG. 19. ¹H NMR spectra [L^{1H3}Co](Cl)₂ in D₂O under anaerobic conditions at 25 C at 0 hr. Paramagnetic peaks are integrated against an internal capillary containing C₆D₆/C₆H₆ (δ 6.62), (80:20).

FIG. 20. ¹H NMR spectra [L^{1H3}Co](Cl)₂ in D₂O under anaerobic conditions at 25 C at 12 days. Paramagnetic peaks are integrated against an internal capillary containing C₆D₆/C₆H₆ (δ 6.51), (80:20). Comparing the 0 hr spectra against the spectra collected after 12 days indicates less than 5% decomposition of the paramagnetic signals when integrated against the internal standard, while an analysis of the “fingerprint” region for L^{1H3} at 9.67 indicates no observable free ligand L^{1H3} in solution.

FIG. 21. Z-Spectra of a 10 mM aqueous sample of [L^{1H3}Fe](OTf)₂ in 100 mM NaCl and 20 mM HEPES buffer at pH 6.8, 7.0, and 7.4 at 37° C.

FIG. 22. Z-Spectra of a 10 mM aqueous sample of [L^{1H3}Fe](Cl)₂ in 100 mM NaCl and 20 mM HEPES buffer at pH 6.8, 7.0, and 7.4 at 37° C.

FIG. 23. CEST spectra for 10 mM aqueous sample of [L^{1H3}Co](Cl)₂ in 100 mM NaCl and 20 mM HEPES buffered at pH of 6.8, 7.0, and 7.4 collected at 37° C.

FIG. 24. Z-Spectra of a 10 mM aqueous sample of [L^{2H3}Fe](OTf)₂ in 100 mM NaCl and 20 mM HEPES buffer at pH 6.8, 7.0, and 7.4 at 37° C.

FIG. 25. Z-Spectra of a 10 mM aqueous sample of [L^{3H3}Fe](OTf)₂ in 100 mM NaCl and 20 mM HEPES buffer at pH 6.8, 7.0, and 7.4 at 37° C.

FIG. 26. Z-Spectra of a 10 mM aqueous sample of [L^{3H3}Fe](OTf)₂ in 100 mM NaCl and 20 mM HEPES buffer at pH 6.8, 7.0, and 7.4 at 25° C.

FIG. 27. CEST spectra for 10 mM aqueous sample of [L^{1H3}Fe](OTf)₂ (L1Fe) in 100 mM NaCl and 20 mM HEPES buffered at pH of 7.4, collected at 25° C. over the course of 7 days to gauge complex stability. No change in CEST efficacy was observed as indicated by the overlapping spectra.

FIG. 28. CEST spectra for 10 mM aqueous sample of [L^{2H3}Fe](OTf)₂ (L2Fe) in 100 mM NaCl and 20 mM HEPES buffered at pH 7.4, collected at 25° C. over the course of 7 days to gauge complex stability. No change in CEST efficacy was observed as indicated by the overlapping spectra.

FIG. 29. CEST spectra for 10 mM aqueous sample of [L^{3H3}Fe](OTf)₂ (L3Fe) in 100 mM NaCl and 20 mM HEPES buffered at pH of 6.8, collected at 25° C. over the course of 7 days to gauge complex stability. No change in CEST efficacy was observed as indicated by the overlapping spectra.

FIG. 30. Aldehyde and carbonyl appended heterocycles to be used to form tripodal Schiff base ligands to support first row transition metal ions to give coordination complexes to act as paraCEST contrast agents.

### DETAILED DESCRIPTION

The present invention refers to the following embodiments:

1 Contrast agent of the formula [N(A₁,A₂,A₃) M](counter ion(s)) for use in a diagnostic method practiced on the human or animal body, wherein
N is a nitrogen atom
M is a divalent metal ion selected from transition metals of the group: V^{II}, Cr^{II}, Fe^{II}, Co^{II}, and Cu^{II};
the counter ion(s) being pharmaceutically acceptable; and wherein
A₁, A₂, and A₃ are independently selected from the group of ligands consisting of:

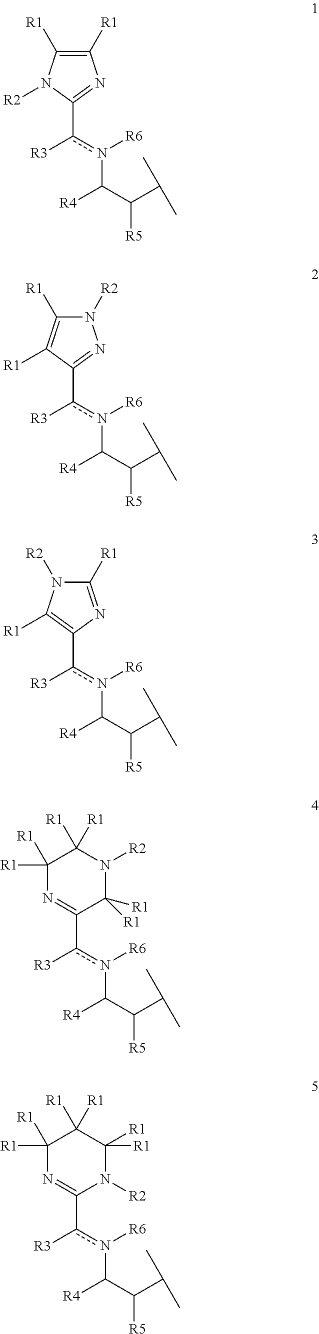

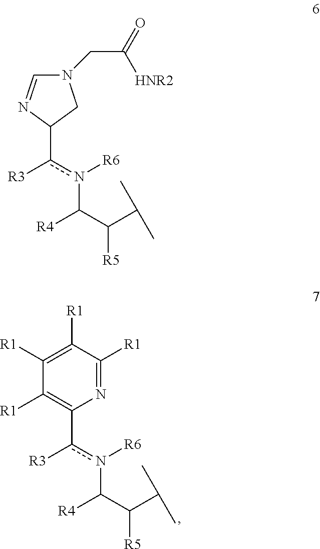

wherein [symbol] denotes a single or a double bond, preferably a double bond, wherein R₆ is absent if there is a double bond; and
R₂, R₃, R₄, R₅, R₆, and each of R₁, are independently selected from the group consisting of: H, OH, SH, CF₃, CN, C(O)NH₂, C(O)H, C(O)OH, halogen (in particular F, Cl, Br, I), optionally substituted C₁₋₄ alkyl, preferably CH₃, C₁₋₄ heteroalkyl, cycloalkyl, C₃₋₇ heterocycloalkyl, C₄₋₁₂ aryl and C₄₋₁₂ heteroaryl groups, R^{k}, SR^{k}, S(O)R^{k}, S(O)₂R^{k}, S(O)OR^{k}, S(O)₂OR^{k}, OS(O)R^{k}, OS(O)₂R^{k}, OS(O)OR^{k}, OS(O)₂R^{k}, OR^{k}, P(O)(OR^{k})(OR^{L}), OP(O)(OR^{k})(OR^{L}), SiR^{k}R^{L}R^{m}, C(O)R^{k}, C(O)OR^{k}, C(O)N(R^{L})R^{k}, OC(O)R^{k}, OC(O)OR^{k}, OC(O)N(R^{k})R^{L},
wherein R, R^{k}, R^{L}, and R^{m} are independently selected from the group consisting of H and optionally substituted C₁₋₄ alkyl, preferably CH₃; C₁₋₄ heteroalkyl, C₃₋₇ cycloalkyl, C₃₋₇ heterocycloalkyl, C₄₋₁₂ aryl, or C₄₋₁₂ heteroaryl groups, wherein two or more of R^{k}, R^{L} and R^{m} may form, together with each other, one or more optionally substituted aliphatic or aromatic carbon cycles or heterocycles; and wherein one or more of R₂, R₃, R₄, R₅, R₆ and each of R₁, can be coupled to a probe, or label (R can e.g., be C₁-C₃ alkyl); and
wherein for ligands 1-6 the following conditions additionally apply:
if R₆ is one of H, OH, NH₂, NHR, SH, C(O)NH₂, C(O)NHR, or C(O)OH, then R₁-R₅ can be independently selected from the group as indicated for R₁-R₅ above;
if R₆ is absent or not one of H, OH, NH₂, NHR, SH, C(O)NH₂, C(O)NHR, or C(O)OH, then R₂ must be H, OH, NH₂, NHR, SH, C(O)NH₂, C(O)NHR, or C(O)OH and/or at least one of R₁, R₃, R₄ and R₅ must be OH, NH₂, NHR, SH, C(O)NH₂, C(O)NHR, or C(O)OH; and
   wherein for ligand 7 the following conditions additionally apply:
if R₆ is one of H, OH, NH₂, NHR, SH, C(O)NH₂, C(O)NHR, or C(O)OH, then R₁, R₃, R₄ and R₅ can be independently selected from the group as indicated for R₁-R₅ above.
if R₆ is absent or R₆ is not one of H, OH, NH₂, NHR, SH, C(O)NH₂, C(O)NHR, or C(O)OH then at least one of R₁, R₃, R₄ and R₅ must be OH, NH₂, NHR, SH, C(O)NH₂, C(O)NHR, or C(O)OH.

In another embodiment, the following conditions apply:

for ligands 1-6 the following conditions additionally apply:
if R₆ is one of H, OH, NH₂, SH, C(O)NH₂, or C(O)OH, then R₁-R₅ can be independently selected from the group as indicated above;
   if R₆ is absent or not one of H, OH, NH₂, SH, C(O)NH₂, or C(O)OH, then R₂ must be H, OH, NH₂, SH, C(O)NH₂, or C(O)OH and/or at least one of R₁, R₃, R₄ and R₅ must be OH, NH₂, SH, C(O)NH₂, or C(O)OH; and
      for ligand 7 the following conditions additionally apply:
if R₆ is one of H, OH, NH₂, SH, C(O)NH₂, or C(O)OH, then R₁, R₃, R₄ and R₅ can be independently selected from the group as indicated above.
if R₆ is absent or R₆ is not one of H, OH, NH₂, SH, C(O)NH₂, or C(O)OH then at least one of R₁, R₃, R₄ and R₅ must be OH, NH₂, SH, C(O)NH₂, or C(O)OH.

In one embodiment, which can be combined with all embodiments described herein, R^{k}, R^{L}, and R^{m} are independently selected from the group consisting of H and optionally substituted C₁₋₄ alkyl, preferably CH₃.

In one embodiment, which can be combined with all embodiments described herein, R₂, R₃, R₄, R₅, and each of R₁, are independently selected from the group consisting of: H, OH, SH, CF₃, CN, C(O)NH₂, C(O)H, C(O)OH, halogen (in particular F, Cl, Br, I), optionally substituted C₁₋₄ alkyl, preferably CH₃, C₁₋₄ heteroalkyl, C₃₋₇ cycloalkyl, C₃₋₇ heterocycloalkyl, C₄₋₁₂ aryl and C₄₋₁₂ heteroaryl groups; wherein one or more of R₂, R₃, R₄, R₅, R₆ and each of R₁, can be coupled to a probe, or label; and wherein the conditions for ligands 1-6 and ligand 7 as specified above additionally apply.

In one embodiment, which can be combined with all embodiments described herein, R₂, R₃, R₄, R₅, and each of R₁, are independently selected from the group consisting of: H, OH, SH, CF₃, CN, C(O)NH₂, C(O)H, C(O)OH, halogen (in particular F, Cl, Br, I), optionally substituted C₁₋₄ alkyl, preferably CH₃; and C₄₋₁₂ aryl; wherein one or more of R₂, R₃, R₄, R₅, R₆ and each of R₁, can be coupled to a probe, or label; and wherein the conditions for ligands 1-6 and ligand 7 as specified above additionally apply.

In the paraCEST compounds of the present invention, the metal atom is in high spin and exhibit 3-fold symmetry in solution. Furthermore, said paraCEST compounds contain paramagnetically shifted labile —OH, —NH, or metal-bound —H₂O protons that undergo exchange with the protons of bulk water. Within the context of the present invention, “halogen” refers to F, Cl, Br, and I.

2. The contrast agent for use according to embodiment 1, which is a magnetic resonance imaging contrast agent, in particular a paraCEST (Chemical Exchange-dependent Saturation Transfer) contrast agent. Preferably, the complexes exhibit paraCEST efficacy at 37° C. of at least 10%, or at least 14%, preferably at least 30% and most preferably at least 45%.
3. The contrast agent for use according to embodiment 1 or 2, wherein the transition metal is Fe^{II}, Co^{II}, and Cu^{II}, in particular Fe^{II} or Co^{II}.
4. The contrast agent for use according to any of the preceding embodiments, wherein the contrast agent is water-soluble. For example, a solution with a concentration of 10 millimolar is sufficient for these compounds 7.4 mg/ml.

The contrast agents also exhibit a high water stability. Stability can be monitored in D₂O by ¹H NMR spectroscopy. Preferably, the contrast agents have a stability of at least 90% or at least 95% over the course of 12 days under anaerobic conditions against an internal standard consisting of a 80%/20% capillary of C₆D₆/C₆H₆.

5. The contrast agent for use according to any of the preceding embodiments, wherein A₁, A₂, and A₃ are the same.
6. The contrast agent for use according to any of the preceding embodiments, wherein the diagnostic method is medical imaging, in particular diagnostic imaging.
7. The contrast agent for use according to any of the preceding embodiments, wherein A1, A2, and A3 are independently selected from the group consisting of:

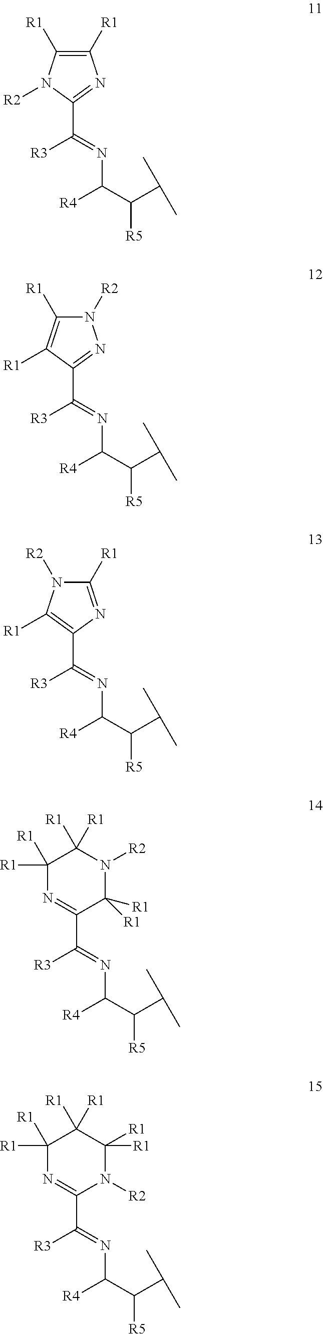

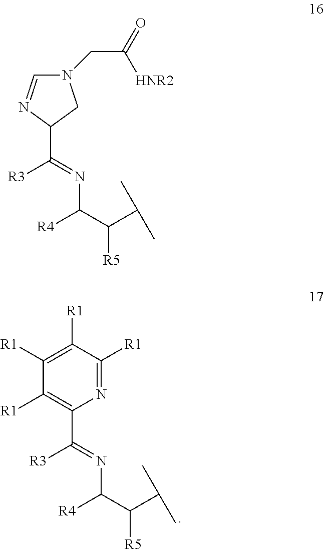

8. The contrast agent for use according to any of the preceding embodiments, wherein the counter ion(s) is/are selected from the group consisting of acetate (OAc⁻), chloride (Cl⁻), iodide (I⁻), bromide (Br⁻), nitrate (NO₃⁻), triflate (OTf⁻) and sulfate (SO₄²⁻). These counter ions are pharmaceutically acceptable.

In general, the pharmaceutically acceptable counteranions to each acid listed in the below table are suitable. Experimentally, the anions of strong acids such as (OTf−) stabilize the compounds towards aerobic oxidation, while the anions from weaker acids (OAc) make these compounds more susceptible towards aerobic oxidation. Accordingly, it is preferred to use anions of strong acids, e.g., anions from strong acids with pKa's less than 4, which provide a higher stability against aerobic/O₂ oxidation:

| | |
|---|---|
| 1-hydroxy-2-naphthoic acid | glycolic acid |
| 2,2-dichloroacetic acid | hippuric acid |
| 2-hydroxyethanesulfonic acid | hydrobromic acid |
| 2-oxoglutaric acid | hydrochloric acid |
| 4-acetamidobenzoic acid | isobutyric acid |
| 4-aminosalicylic acid | lactic acid (DL) |
| acetic acid | lactobionic acid |
| adipic acid | lauric acid |
| ascorbic acid (L) | maleic acid |
| aspartic acid (L) | malic acid (−L) |
| benzenesulfonic acid | malonic acid |
| benzoic acid | mandelic acid (DL) |
| camphoric acid (+) | methanesulfonic acid |
| camphor-10-sulfonic acid (+) | naphthalene-1,5-disulfonic acid |
| capric acid (decanoic acid) | naphthalene-2-sulfonic acid |
| caproic acid (hexanoic acid) | nicotinic acid |
| caprylic acid (octanoic acid) | nitric acid |
| carbonic acid | oleic acid |
| cinnamic acid | oxalic acid |
| citric acid | palmitic acid |
| cyclamic acid | pamoic acid |
| dodecylsulfuric acid | phosphoric acid |
| ethane-1,2-disulfonic acid | proprionic acid |
| ethanesulfonic acid | pyroglutamic acid (−L) |
| formic acid | salicylic acid |
| fumaric acid | sebacic acid |
| galactaric acid | stearic acid |
| gentisic acid | succinic acid |
| glucoheptonic acid (D) | sulfuric acid |
| gluconic acid (D) | tartaric acid (+L) |
| glucuronic acid (D) | thiocyanic acid |
| glutamic acid | toluenesulfonic acid (p) |
| glutaric acid | undecylenic acid |
| glycerophosphoric acid | |

9. The contrast agent for use according to any of the preceding embodiments, wherein R₁, R₄, R₅, and R₆ are independently selected from the group consisting of: H, F, Cl, Br, I methyl, OMe, OH, and CF₃, wherein R₂ is H, or OH; wherein preferably R₂, R₄, R₅, and R₆ are H, and R₃═H or CH₃.
10. The contrast agent for use according to any of the preceding embodiments, wherein R₁, R₂, R₃, R₄, R₅, and R₆ are independently selected from the group consisting of: H, OH, SH, CF₃, CN, halogen, optionally substituted C₁₋₄ alkyl, C₁₋₄ heteroalkyl, C₃₋₇ cycloalkyl, C₃₋₇ heterocycloalkyl, C₄₋₁₂ aryl C(O)NH₂ or C(O)OH and C₄₋₁₂ heteroaryl groups.
wherein R₆ is H, OH, or an C₁₋₄ alkyl; and wherein if R² does not equal H, OH, SH, C(O)NH₂, or C(O)OH, at least one of R₁, R₃, R₄, and R₅ must equal OH, SH, NH₂, C(O)NH₂, C(O)OH.
11. The contrast agent for use according to any of the preceding embodiments, wherein R₁═H, F, Cl, Br, I, CH₃, OCH₃, OH or CF₃; R₂, R₄, R₅, and R₆═H; and R₃═H or CH₃.
12. The contrast agent for use according to any of the preceding embodiments, wherein at least one of R₁═OH, preferably wherein one of R₁═OH and the remaining R₁═H; R₂, R₄, R₅, and R₆═H; and R₃═H or CH₃.
13. The contrast agent for use according to any of the preceding embodiments, wherein one or more of R₂, R₃, R₄, R₅, R₆ and each of R₁, preferably one of R₂, R₃, R₄, and R₆ is coupled to a probe, or label, wherein the probe or label can be an antibody, peptide such as an oligopeptide, e.g., being comprised of 3-20 amino acids, or a dye, such as a fluorescent compound, and ¹⁹F-based probe.
14. The contrast agent for use according to any of the preceding embodiments, wherein A₁, A₂, and A₃ are selected from

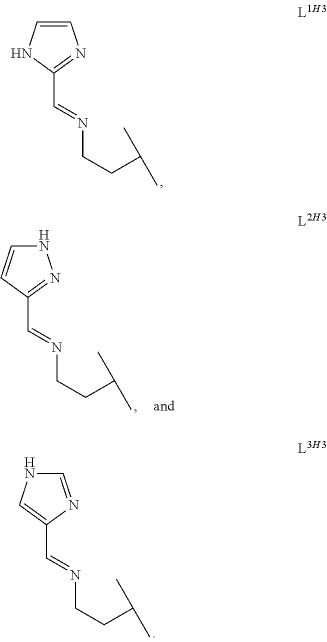

15. The contrast agent for use according to embodiment 14, wherein the counter ion is trifluoromethanesulfonate or chloride and the metal is Fe or Co or Ni, in particular, the contrast agent is selected from the group consisting of [L^{1H3} Co](counter ion(s)), [L^{2H3} Co](counter ion(s)), [L^{3H3} Co](counter ion(s)), [L^{1H3} Fe](counter ion(s)), [L^{2H3} Fe](counter ion(s)), [L^{3H3} Fe](counter ion(s)), [L^{1H3} Ni](counter ion(s)), [L^{2H3} Ni](counter ion(s)), and [L^{3H3} Ni](counter ion(s)), further preferred [L^{1H3} Co](OTf)₂, [L^{2H3} Co](OTf)₂, [L^{3H3} Co](OTf)₂, [L^{1H3} Fe](OTf)₂, [L^{2H3} Fe](OTf)₂, [L^{3H3} Fe](OTf)₂, [L^{1H3} Ni](OTf)₂, [L^{2H3} Ni](OTf)₂, [L^{3H3} Ni](OTf)₂, [L^{1H3} Co](Cl)₂, [L^{2H3} Co](Cl)₂, [L^{3H3} Co](Cl)₂, [L^{1H3} Fe](Cl)₂, [L^{2H3} Fe](Cl)₂, [L^{3H3} Fe](Cl)₂, [L^{1H3} Ni](Cl)₂, [L^{2H3} Ni](Cl)₂, and [L^{3H3} Ni](Cl)₂.
16. A contrast agent as defined in any of embodiments 1-17, preferably wherein the pharmaceutically acceptable counterion(s) is/are as defined in embodiment 8.
17. A pharmaceutical composition comprising a contrast agent as defined in any of embodiments 1 to 16 and at least one pharmaceutically acceptable excipient.
18. A pharmaceutical composition as defined in embodiment 17 for use as a medicament.
19. A method of in vitro medical imaging, especially of diagnostic imaging, comprising administering a compound as defined in any of embodiments 1 to 16 to a sample.

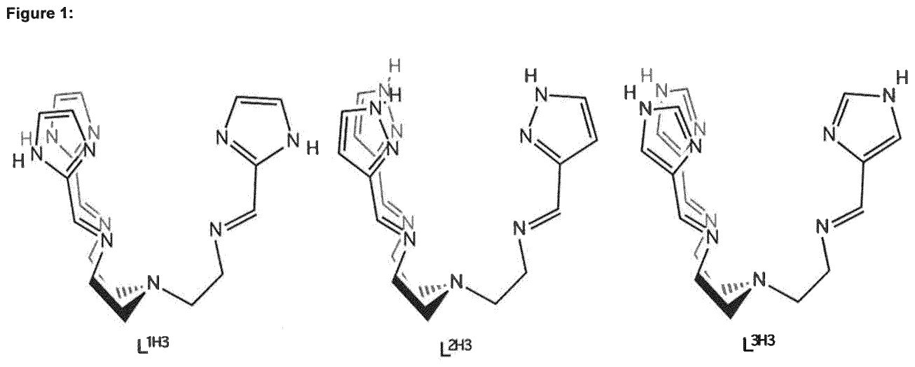

Ligands L^{1H3}, L^{2H3}, and L^{3H3} (FIG. 1) were prepared as described in the example section.

Ligand L^{1H3} corresponds A₁/A₂/A₃ as defined in the claims, wherein ligand 1 is used, and wherein R₂, R₃, R₄, R₅, and each of R₁═H.

Ligand L^{2H3} corresponds A₁/A₂/A₃ as defined in the claims, wherein ligand 2 is used, and wherein R₂, R₃, R₄, R₅, and each of R₁═H.

Ligand L^{3H3} corresponds A₁/A₂/A₃ as defined in the claims, wherein ligand 3 is used, and wherein R₂, R₃, R₄, R₅, and each of R₁═H.

The metalation of ligands L^{1H3}, L^{2H3}, and L^{3H3} was straightforward, providing dicationic coordination complexes when combined with M^{II} ion (M=Fe^{II}(OTf)₂, Fe^{II}Cl₂, or Co^{II}Cl₂). The method is broadly applicable giving the coordination complexes [L^{1H3}Fe^{II}](OTf)₂, [L^{1H3}Fe^{II}](Cl)₂, [L^{1H3}Co^{II}](Cl)₂, [L^{2H3}Fe^{II}](OTf)₂, and [L^{3H3}Fe^{II}](OTf)₂.

The neutral six-coordinate tripodal Schiff base ligands L^{1H3} L^{2H3}, and L^{3H3} (FIG. 1) were previously reported to support high-spin Fe^{II} as perchlorate salts with the general formulation [LFe]²⁺(ClO₄)₂. (cf. Brewer, C.; Brewer, G.; Luckett, C.; Marbury, G. S.; Viragh, C.; Beatty, A. M.; Scheidt, W. R. Inorg Chem 2004, 43 (7), 2402-2415; Hardie, M. J.; Kilner, C. A.; Halcrow, M. A. Acta Cryst (2004). C60, 177-179 [doi:10.1107/S010827010400407X] 2004, 1-10).

The ligands L^{1H3}, L^{2H3}, and L^{3H3} have been examined for their ability to form analogous water-soluble dicationic first row transition metal complexes as triflate (OTf) or chloride (co salts with the metals Fe and Co. In specific, the complexes [L^{1H3}Fe](OTf)₂, [L^{1H3}Fe](Cl)₂, [L^{1H3}Co](Cl)₂, [L^{2H3}Fe](OTf)₂, and [L^{3H3}Fe](OTf)₂ have been synthesized, characterized in the solution-state, and studied in relation to their paraCEST efficacy through the compilation of Z-spectra. Solution-state ¹H and ¹⁹F NMR spectra for many of the complexes are presented here for the first time. The reported family of isostructural six-coordinate tripodal Schiff base ligands (L^{1H3}, L^{2H3}, and L^{3H3}) supports Feⁿ as dicationic triflate and chloride salts and Co^{II} as a chloride salt. All complexes exhibit 3-fold symmetry in solution, and ¹⁹F NMR reveal non-coordinating triflate anions for [L^{1H3}Fe](OTf)₂, [L^{2H3}Fe](OTf)₂, and [L^{3H3}Fe](OTf)₂ in CD₃CN and CD₃OD.

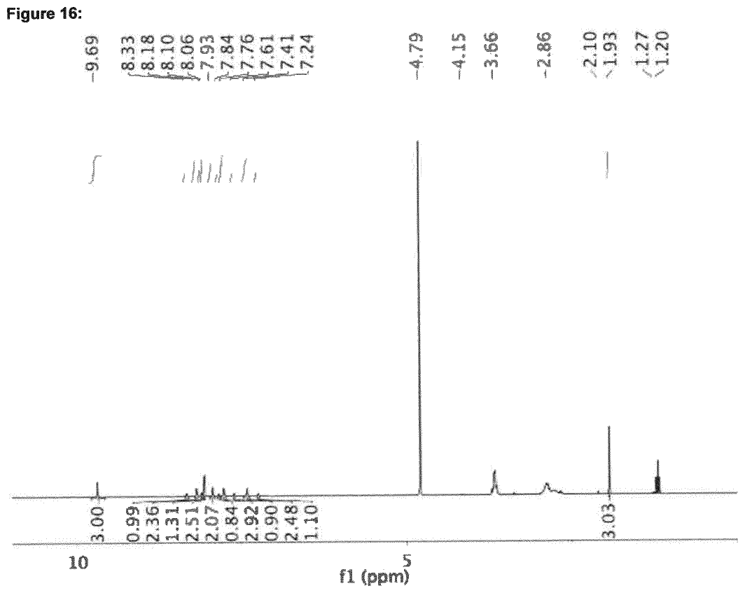

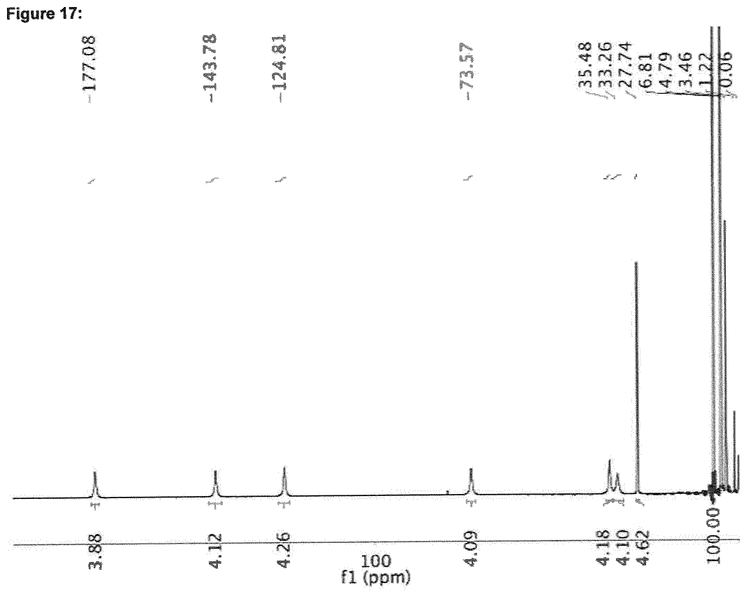

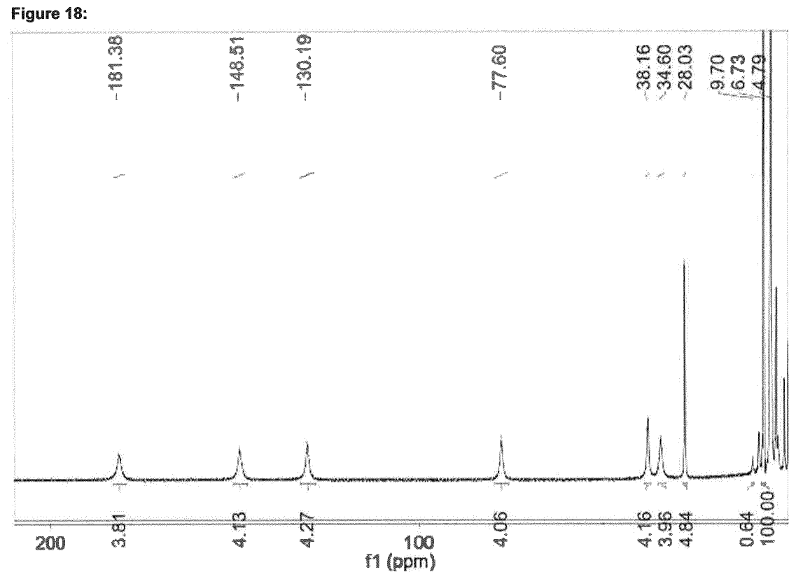

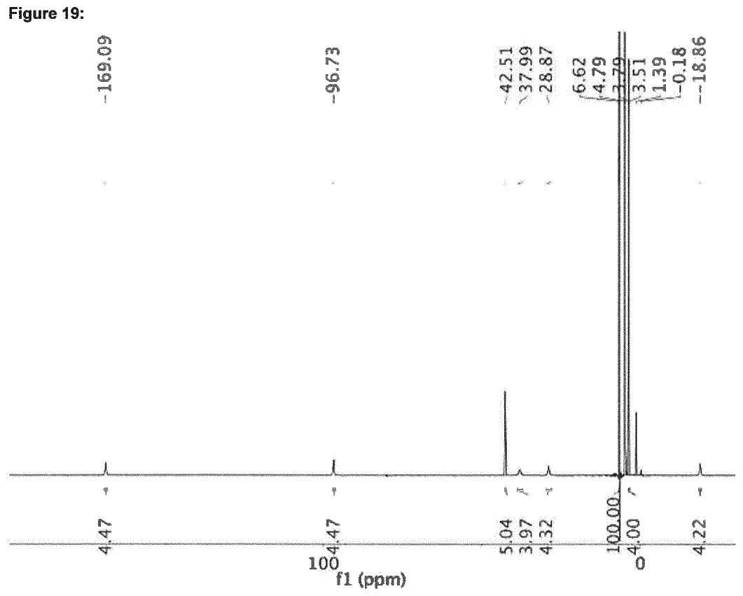

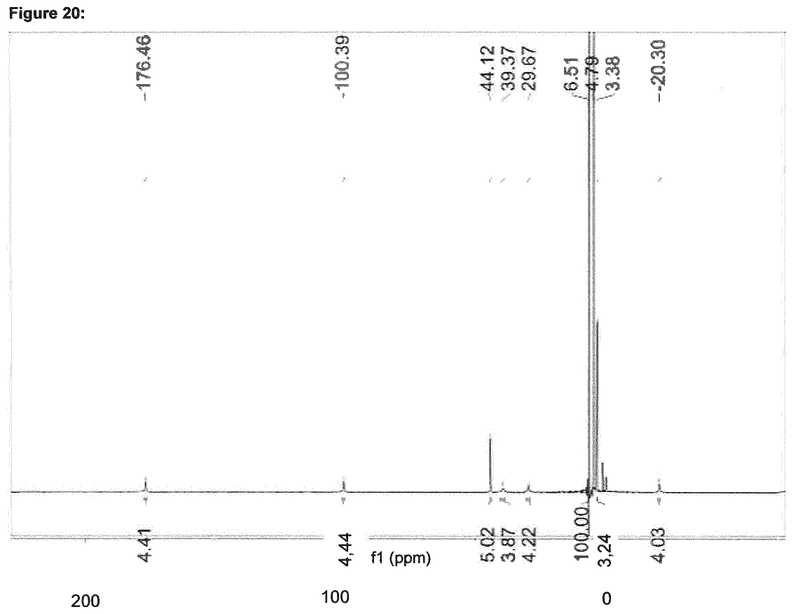

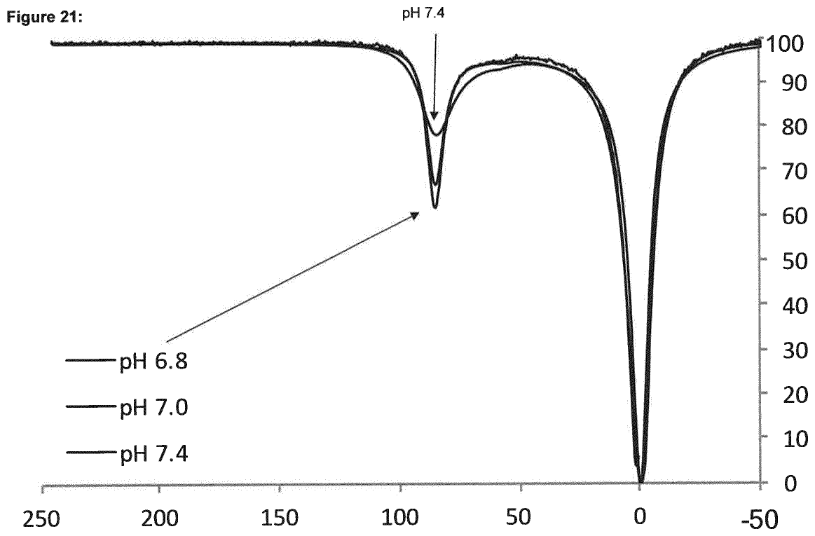

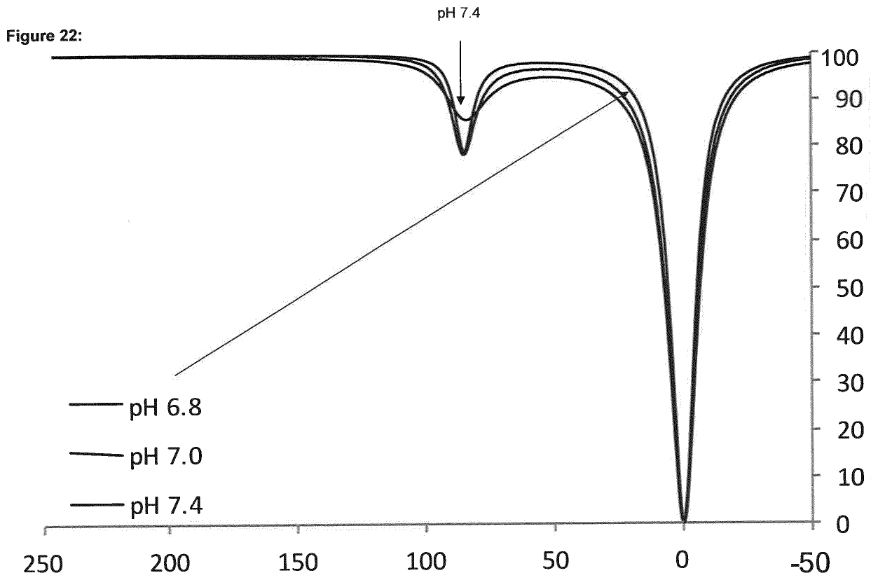

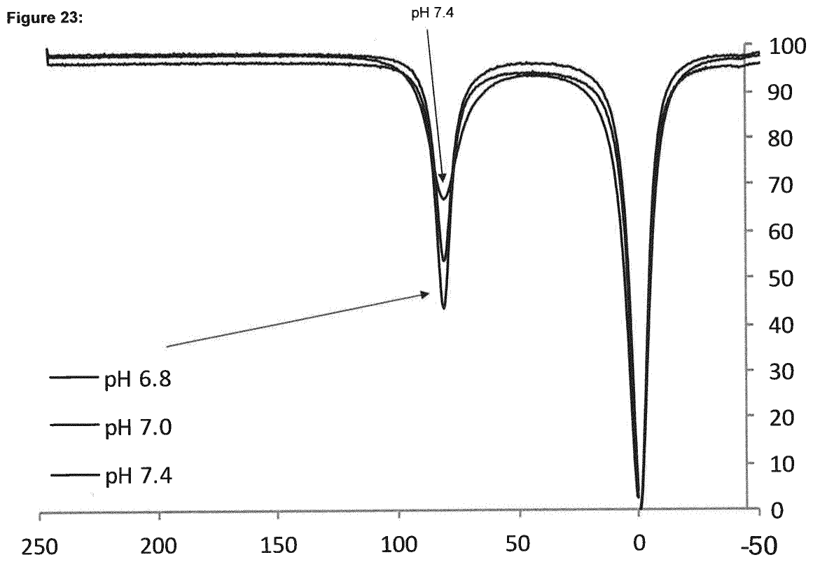

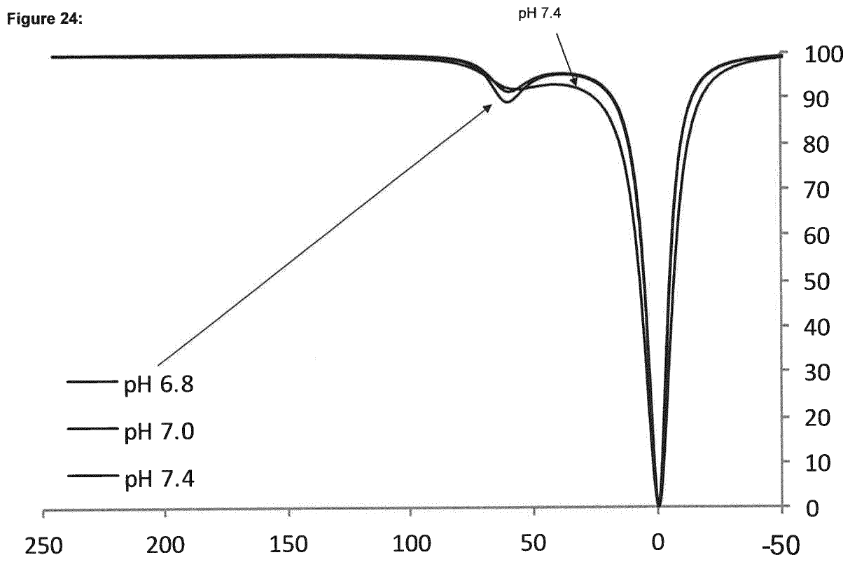

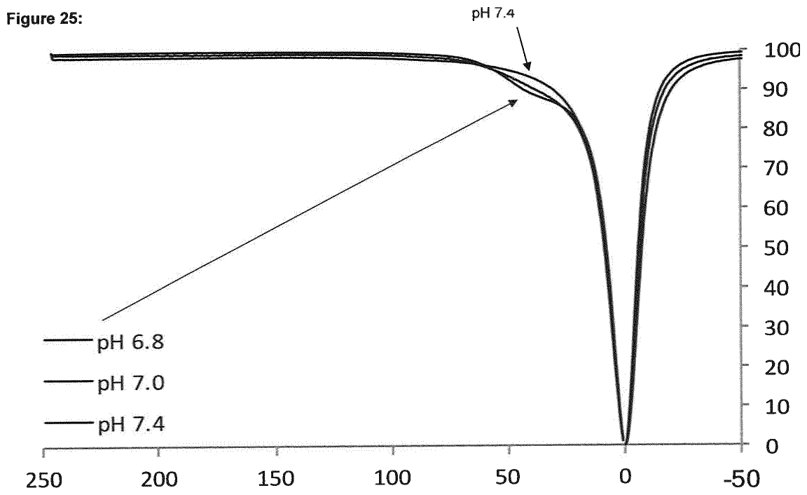

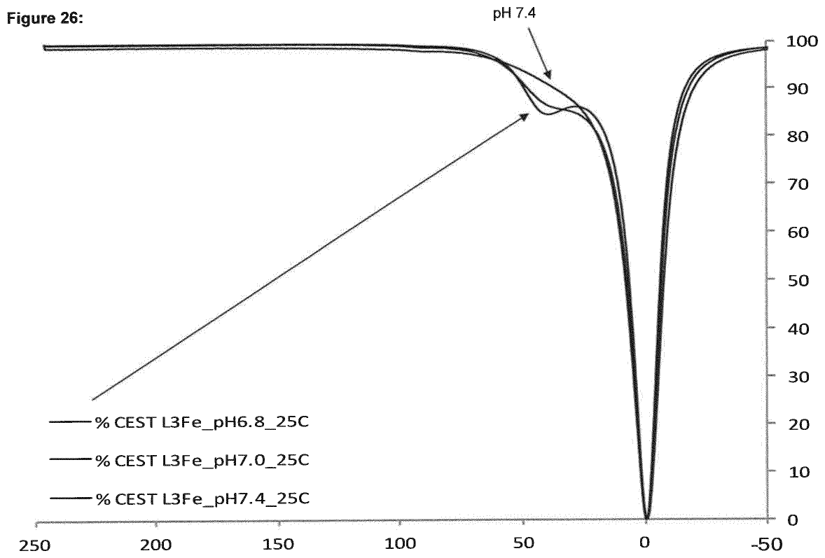

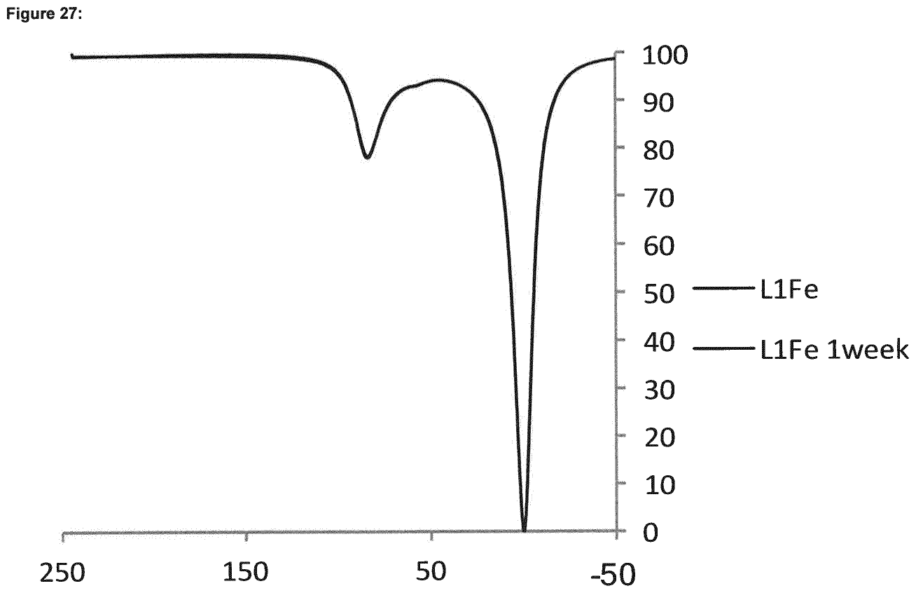

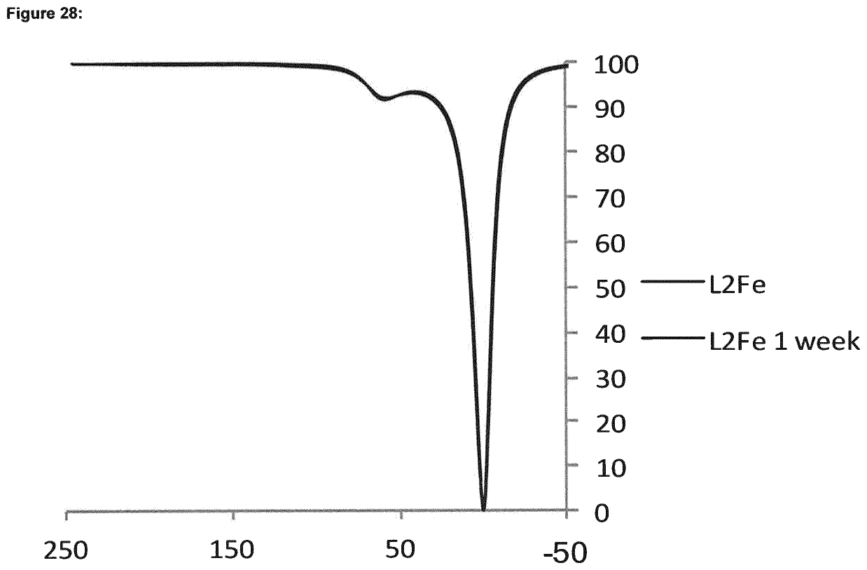

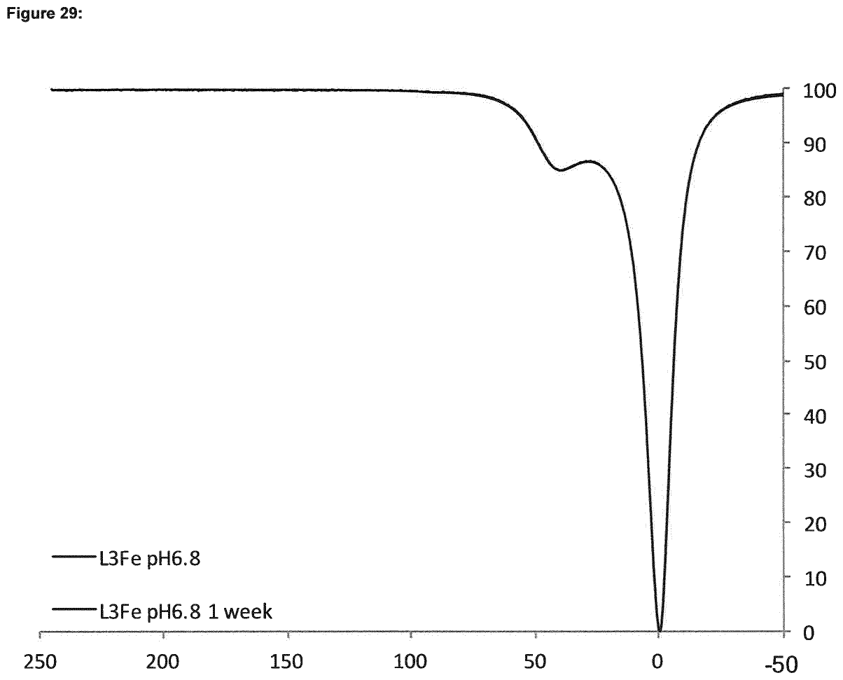

ParaCEST efficacy experiments were conducted and demonstrate that these agents exhibit paramagnetic chemical exchange saturation transfer in aqueous solution buffered to physiologically relevant pH and ionic strength under anaerobic conditions (FIGS. 21 to 26). These results demonstrate proof-of-principle that these types of complexes can act as paraCEST contrast agents in a biological setting. Surprisingly, Z-spectra collected at the acidic pH of diseased tissue (pH 6.8) provide greater contrast (FIG. 21-26) than when at the neutral or the slightly alkaline pH of healthy tissue (pH 7.0, 7.4), thus providing paraCEST agents that are “activated” by the acidic pH associated with diseased tissue (FIGS. 21-26). Furthermore, these complexes maintain their contrast efficacy in anaerobic aqueous solution for at least a week in solutions of physiologically relevant pH and ionic strength (selected stability experiments presented in FIGS. 27, 28 and 29). Additionally, the stability of complexes [L^{1H3}Fe](OTf)₂ and [L^{1H3}Co](Cl)₂ were monitored in D₂O by ¹H NMR spectroscopy over the course of 12 days under anaerobic conditions against an internal standard consisting of a 80%/20% capillary of C₆D₆/C₆H₈, revealing very little decomposition (less than 5%) or formation of free ligand over the course of 12 days (FIGS. 16-20).

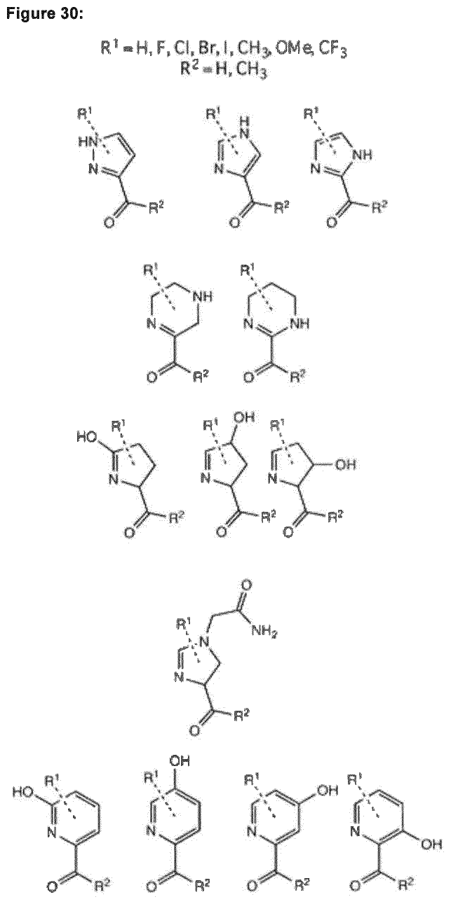

These results lead to a number of rational conclusions. 1) The use of heterocycles appended with aldehyde or carbonyl functionalities (see FIG. 30, R²═H, Me), when combined with 0.33 molar equivalents of tris-(2-aminoethyl)amine will allow the synthesis of hexacoordinate tripodal Schiff-base ligands that are anticipated to act in an analogous fashion to L^{1H3}, L^{2H3}, and L^{3H3} to provide ligands that will coordinate transition metals and give complexes that exhibit paraCEST efficacy. 2) Tripodal Schiff-base ligands formed from heterocycles appended with aldehydes or carbonyl functionalities, that are further functionalized at the heterocycle with electron donating or withdrawing substituents (FIG. 30, R²═H, Me; R¹═H, F, Cl, Br, I, CH₃, OCH₃, CF₃) will alter the electronics of the ligand, and the stability of the metal complexes towards oxidative degradation (FIG. 30). 3) Tripodal Schiff-base ligands formed from heterocycles appended with aldehydes or carbonyl functionalities, that are further functionalized at the heterocycle with electron donating or withdrawing substituents (FIG. 30, R²═H, Me; R¹═H, F, Cl, Br, I, CH₃, OCH₃, CF₃) will support first-row transition metals to provide M^{II} complexes (M=Fe^{II}, Co^{II}, and Ni^{II}) that act to provide contrast in MRI through paramagnetic chemical exchange saturation transfer. 4) Tripodal Schiff-base ligands formed from heterocycles appended with aldehydes or carbonyl functionalities, that are further functionalized at the heterocycle with electron donating or withdrawing substituents (FIG. 30, R²═H, Me; R¹═H, F, Cl, Br, I, CH₃, OCH₃, CF₃) will be able to support the first row transition metals Fe^{II}, Co^{II}, and Ni^{II} as acetate (OAc⁻), bromide (Br⁻), chloride (Cl⁻), iodide (I⁻), nitrate (NO₃⁻), triflate (OTf⁻), and sulfate (SO₄²⁻) salts. These complexes can be anticipated to exhibit varying degrees of solubility in H₂O, CH₃OH, and CH₃CN, allowing their solution-state structures to be determined and paraCEST efficacy studied.

Methods

General Experimental Procedures

All reactions and subsequent manipulations were performed under anaerobic and anhydrous conditions under an atmosphere of nitrogen or argon in an MBraun glovebox or using Schlenk techniques unless otherwise noted. Diethyl ether was dried over sodium benzophenone ketyl and distilled under a 1.2 atm dynamic argon flow directly into solvent transfer flasks that had undergone three vacuum-argon-purge cycles on a high-vacuum Schlenk line prior to solvent transfer, thus ensuring transfer of anhydrous and anaerobic solvent for glovebox use. Likewise MeCN, and MeOH were dried over CaH₂ and distilled under a 1.2 atm dynamic argon flow directly into solvent transfer flasks as outlined above for glovebox use. All glovebox solvents were stored over 10% by mass activated 3 Å molecular sieves for a minimum of 24 h before use.

CD₃CN and CD₃OD were transferred into the glovebox as received and stored over 10% mass of 3 Å molecular sieves for 48 h prior to use. All other reagents were purchased from commercial suppliers and used as received. All NMR experiments were conducted at 25° C. unless otherwise noted. ¹H NMR and ¹⁹F NMR spectra were recorded on a Bruker AV 401 and Bruker AV301 instrument, respectively. ¹H and ¹⁹F{¹H} NMR spectra are referenced to external SiMe₄ and CFCl₃ using the unified xi scale (¹⁹F freq CFCl₃/1H freq TMS=0.9409011). Ligands L^{1H3}, L^{2H3}, and L^{3H3} were synthesized and purified based upon previously reported procedures.

CEST Spectroscopy (Z-spectra) CEST experiments were conducted on a 9.4 T NMR spectrometer through a presaturation experiment plotted as normalized water signal intensity (M_{z}/M₀%) against frequency offset (ppm) in 0.5 ppm increments. A presaturation pulse power (B₁) of 18.7 μT was applied for 2 seconds at either 25° C. or 37° C. The 2-D array of spectra are analyzed with the MestReNova software package and an integral table compiled containing the integration of the H₂O resonance centered at 4.79 ppm from 5.4 to 4.2 ppm as a function of presaturation frequency. This data is in-turn used to compile a plot of the signal intensity of bulk water M_{z}/M₀ as a function of presaturation frequency, which indicates the paramagnetic presaturation frequency that results in the greatest reduction in signal arising from bulk water. These experiments are conducted by preparing a 10 mM solution of the appropriate complex in an anaerobic aqueous stock solution buffered to relevant physiological pH (6.8, 7.0, 7.4) and a physiologically relevant ionic strength using 20 mM HEPES buffer and 100 mM NaCl in a J-Young NMR tube. A D₂O capillary is inserted into the J-Young NMR tube to provide a deuterium “lock” signal.

### EXAMPLES

The following examples describe the present invention in detail, but they are not to be construed to be in any way limiting for the present invention.

### Example 1: Synthesis of Ligands L^{1H3}, L^{2H3}, and L^{3H3} (FIG. 1)

Ligands L^{1H3}, L^{2H3}, and L^{3H3} were synthesized and purified based upon previously reported procedures.

Ligands L^{1H3}, L^{2H3}, and L^{3H3} were prepared by the condensation of one equivalent of tris-(2-aminoethyl)amine with 3.05 equivalents of the appropriate imidazole or pyrazole-bearing aldehydes in refluxing anhydrous methanol under an aerobic atmosphere in an adaptation of previously described procedures (cf. Brewer, C.; Brewer, G.; Luckett, C.; Marbury, G. S.; Viragh, C.; Beatty, A. M.; Scheidt, W. R. Inorg Chem 2004, 43 (7), 2402-2415; Hardie, M. J.; Kilner, C. A.; Halcrow, M. A. Acta Cryst (2004). C60, 177-179 [doi:10.1107/S010827010400407X] 2004, 1-10). Reduction of solvent volume, followed by trituration with ethyl acetate resulted in precipitation of the off-white-to-yellow solids L^{1H3}, L^{2H3}, and L^{3H3}, which were isolated by vacuum filtration and dried under vacuum at 100° C. to afford nearly quantitative yields. Metalation of ligands L^{1H3}, L^{2H3}, and L^{3H3} was straightforward, providing dicationic coordination complexes when an equivalent of M^{II} ion (M=Fe^{II}(OTf)₂, Fe^{II}Cl₂, or Co^{II}Cl₂) was combined with an equivalent of ligand in anhydrous methanol under anaerobic conditions, and refluxed for an hour. This method was broadly applicable giving the coordination complexes [L^{1H3}Fe^{II}](OTf)₂, [L^{1H3}Fe^{II}](Cl)₂, [L^{1H3}Co^{II}](Cl)₂, [L^{2H3}Fe^{II}](OTf)₂, and [L^{3H3}Fe^{II}](OTf)₂. Nearly quantitative yields of a fine powder precipitate can be isolated by layering a methanol solution of a dicationic complex with diethyl ether and storing for 48 h in a glovebox freezer (−35° C.), followed by removal of solvent by decantation, and drying in vacuo. The solution state structures of compounds [L^{1H3}Fe^{II}](OTf)₂, [L^{1H3}Fe^{II}](Cl)₂, [L^{1H3}Co^{II}](Cl)₂, [L^{2H3}Fe^{II}](OTf)₂, and [L^{3H3}Fe^{II}](OTf)₂ are all consistent with 3-fold symmetry in solution, but as is the case with most paramagnetic ¹H NMR spectra individual ¹H resonances can not be definitively assigned without costly isotopic labeling experiments.

The ligands L^{1H3}, L^{2H3}, and L^{3H3} have been examined for their ability to form analogous water-soluble dicationic first row transition metal complexes as triflate (OTf) or chloride (co salts with the metals Fe and Co. In specific, the complexes [L^{1H3}Fe^{II}](OTf)₂, [L^{1H3}Fe^{II}](Cl)₂, [L^{1H3}Co^{II}](Cl)₂, [L^{2H3}Fe^{II}](OTf)₂, and [L^{3H3}Fe^{II}](OTf)₂ have been synthesized, characterized in the solution-state, and studied in relation to their paraCEST efficacy through the compilation of Z-spectra. Solution-state ¹H and ¹⁹F NMR spectra for many of the complexes are presented here for the first time. The reported family of isostructural six-coordinate tripodal Schiff base ligands (L^{1H3}, L^{2H3}, and L^{3H3}) supports Fe^{II} as dicationic triflate and chloride salts and Co^{II} as a chloride salt. All complexes exhibit 3-fold symmetry in solution, and ¹⁹F NMR reveal non-coordinating triflate anions for [L^{1H3}Fe](OTf)₂, [L^{2H3}Fe](OTf)₂, and [L^{3H3}Fe](OTf)₂ in CD₃CN and CD₃OD.

The paraCEST efficacy experiments were conducted at concentrations of 10 mM of metal complex and demonstrate that these agents exhibit paramagnetic chemical exchange saturation transfer in aqueous solution buffered to physiologically relevant pH and ionic strength under anaerobic conditions (FIGS. 21 to 26). These results demonstrate proof-of-principle that these types of complexes can act as paraCEST contrast agents in a biological setting.

Interestingly, Z-spectra collected at the acidic pH of diseased tissue (pH 6.8) provide greater contrast (FIGS. 21-26) than when at the neutral or the slightly alkaline pH of healthy tissue (pH 7.0, 7.4), thus providing paraCEST agents that are “activated” by the acidic pH associated with diseased tissue (FIGS. 21-26). Furthermore, these complexes maintain their contrast efficacy in anaerobic aqueous solution for at least a week in solutions of physiologically relevant pH and ionic strength (selected stability experiments presented in FIGS. 27-29). Additionally, the stability of complexes [L^{1H3}Fe](OTf)₂ and [L^{1H3}Co](Cl)₂ were monitored in D₂O by ¹H NMR spectroscopy over the course of 12 days under anaerobic conditions against an internal standard consisting of a 80%/20% capillary of C₆D₆/C₆H₆, revealing very little decomposition (less than 5%) or formation of free ligand over the course of 12 days (FIGS. 16-20).

### Example 2: Synthesis of [L^{1H3}Fe](OTf)₂

Fe(OTf)₂ (524 mg, 1.46 mmol) and L^{1H3} (557 mg, 1.46 mmol) were combined in a 100 ml Schlenk tube in anhydrous MeOH (10 ml) under anaerobic conditions in a glovebox, sealed and refluxed for 1 h before removal of solvent in vacuo using Schlenk techniques. The resulting orange solid was dissolved in minimal anhydrous MeOH inside a glovebox, passed through a filter with celite pad and subsequently layered with Et₂O and stored at −35° C. for 48 h, providing a fine orange powder in nearly quantitative yields (1.05 g, 1.4 mmol, 98%). ¹H NMR (400 MHz, 25° C., CD₃CN): δ 184.7 (s, 4H); 153.6 (s, 3H); 129.8 (s, 3H); 95.7 (s, 3H); 80.7 (s, 3H); 39.3 (s, 3H); 36.3 (s, 3H); 28.8 (s, 3H). ¹⁹F NMR (400 MHz, 25° C., CD₃CN): δ 78.2. ¹H NMR (400 MHz, 25° C., CD₃OD): δ 181.1 (s, 4H); 153.7 (s, 3H); 124.5 (s, 3H); 80.1 (s, 3H); 39.0 (s, 3H); 37.1 (s, 31-1); 28.2 (s, 3H). ¹⁹F NMR (300 MHz, 25° C., CD₃CN): δ 79.6.

### Example 3: Synthesis of L^{1H3}FeCl₂

FeCl₂THF_{1.5} (57 mg, 0.242 mmol) and L^{1H3} (90 mg, 0.242 mmol) were combined in a 100 ml Schlenk tube in anhydrous MeOH (10 ml) under anaerobic conditions in a glovebox, sealed and refluxed for 1 h before removal of solvent in vacuo using Schlenk techniques. The resulting orange solid was dissolved in minimal anhydrous MeOH inside a glovebox, passed through a filter with celite pad and subsequently layered with Et₂O and stored at −35° C. for 48 h, providing a fine orange solid in good yields (0.105 g, 0.207 mmol, 85%). ¹H NMR (400 MHz, 25° C., CD₃OD): δ): δ 181.1 (s, 3H); 153.6 (s, 3H); 124.6 (s, 3H); 80.2 (s, 3H); 39.0 (s, 3H); 37.1 (s, 3H); 28.2 (s, 3H). L^{1H3}FeCl₂ was found to be insoluble in CD₃CN.

### Example 4: Synthesis of [L^{1H3}Co](Cl)₂

CoCl₂ (130 mg, 1.00 mmol) and L^{1H3} (380 mg, 1.00 mmol) were combined in a 100 ml Schlenk tube in anhydrous MeOH (10 ml) under anaerobic conditions in a glovebox, sealed and refluxed for 1 h before removal of solvent in vacuo using Schlenk techniques. The resulting beige solid was dissolved in minimal anhydrous MeOH inside a glovebox, passed through a filter with celite pad and subsequently layered with Et₂O and stored at −35° C. for 48 h, providing a fine beige powder in nearly quantitative yields (0.505 g, 0.989 mmol, 98%). ¹H NMR (400 MHz, 25° C., CD₃OD: δ 170.8 (s, 3H); 81.5 (s, 3H); 43.6 (s, 3H); 36.9 (s, 3H); 26.5 (s, 3H); 3.35 (s, 3H); −22.7 (s, 3H). L^{1H3}CoCl₂ was found to be insoluble in CD₃CN.

### Example 5: Synthesis of [L^{2H3}Fe](OTf)₂

Fe(OTf)₂ (850 mg, 2.23 mmol) and L^{2H3} (800 mg, 2.23 mmol) were combined in a 100 ml Schlenk tube in anhydrous MeOH (10 ml) under anaerobic conditions in a glovebox, sealed and refluxed for 1 h before removal of solvent in vacuo using Schlenk techniques. The resulting orange solid was dissolved in minimal anhydrous MeOH inside a glovebox, passed through a filter with celite pad and subsequently layered with Et₂O and stored at −35° C. for 48 h, providing a fine orange powder in nearly quantitative yields (1.58 g, 2.15 mmol, 96%). ¹H NMR (400 MHz, 25° C., CD₃CN): δ 226.8 (s, 3H); 159.7 (s, 3H); 154.5 (s, 3H); 79.9 (s, 3H); 74.5 (s, 3H); 38.6 (s, 3H); 36.5 (s, 3H); 27.2 (s, 3H). ¹⁹F NMR (300 MHz, 25° C., CD₃CN): δ 77.0 (s, 3F)¹H NMR (400 MHz, 25° C., CD₃OD): δ 218.2 (s, 3H); 153.5 (s, 3H); 145.8 (s, 3H); 78.0 (s, 3H); 37.6 (s, 6H); 26.3 (s, 3H). ¹⁹F NMR (300 MHz, 25° C., CD₃OD): δ 79.7 (s, 3F).

### Example 6: Synthesis of [L^{3H3}Fe](OTf)₂

Fe(OTf)₂ (531 mg, 1.5 mmol) and L^{3H3} (570 mg, 1.5 mmol) were combined in a 100 ml Schlenk tube in anhydrous MeOH (10 ml) under anaerobic conditions in a glovebox, sealed and refluxed for 1 h before removal of solvent in vacuo using Schlenk techniques. The resulting purple solid was dissolved in minimal anhydrous MeOH inside a glovebox, passed through a filter with celite pad and subsequently layered with Et₂O and stored at −35° C. for 48 h, providing a fine purple powder in nearly quantitative yields (1.05 g, 1.42 mmol, 94%). ¹H NMR (400 MHz, 25° C., CD₃CN): δ 165.5 (s, 3H); 151.8 (s, 3H); 137.2 (s, 3H); 94.9 (s, 3H); 72.7 (s, 3H); 51.9 (s, 3H); 39.6 (s, 3H); 35.5 (s, 3H). ¹⁹F NMR (300 MHz, 25° C., CD₃CN): δ 79.5 (s, 3F)¹H NMR (400 MHz, 25° C., CD₃OD): δ 161.0 (s, 3H); 149.8 (5, 3H); 132.2 (s, 3H); 95.0 (s, 3H); 71.3 (s, 3H); 39.6 (s, 3H); 36.1 (s, 3H). ¹⁹F NMR (300 MHz, 25° C., CD₃OD): δ 80.0 (s, 3F).

### CITED LITERATURE

Du, K.; Harris, T. D. J. Am. Chem. Soc. 2016, 138 (25), 7804-7807.
Jeon, I.-R.; Park, J. G.; Haney, C. R.; Harris, T. D. Chem. Sci. 2014, 5 (6), 2461-2465.
Brewer, C.; Brewer, G.; Luckett, C.; Marbury, G. S.; Viragh, C.; Beatty, A. M.; Scheidt, W. R. Inorg Chem 2004, 43 (7), 2402-2415
Hardie, M. J.; Kilner, C. A.; Halcrow, M. A. Acta Cryst (2004). C60, 177-179 [doi:10.1107/S010827010400407X] 2004, 1-10.
Acta Crystallographica 2004, C60, m177-m179, Hardie et al.; “{Tris[4-(1H-pyrazol-3-yl)-3-azabut-3-enyl]amine}iron(II) diperchlorate monohydrate” discloses ligands, however not in the context of MRI.
Brewer et al., Dalton Transactions March 2006, 5617-5629; “A DFT computational study of spin crossover in iron (III) and iron (II) tripodal imidazole complexes. A comparison of . . . ”.
Brewer et al., Dalton Transactions February 2006; 28(8), 1009-1019;
“Synthesis and characterization of manganese(II) and iron(III) d⁵ tripodal imidazole complexes. Effect of oxidation state, protonation state and ligand conformation on coordination number and spin state”.
Brewer et al., Inorg Chem. 2004, Apr. 5; 43(7), 2402-2415 “Proton Contol of Oxidation and Spin State in a Series of Iron Tripodal Imidazole Complexes”.
U.S. Pat. No. 5,820,851A.
U.S. Pat. No. 5,869,026
WO 2006/114739A2.
WO2009/072079A2
WO 2009/126289A2
WO 2012/155076A2
WO2012/155085A1
JP2000119247
JPH01272568

1.-18. (canceled)

19. A method of diagnostic imaging which comprises administering a patient a contrast agent according claim 34.

20. The method according to claim 19, wherein the contrast agent is a magnetic resonance imaging contrast agent.

21. The method according to claim 19, wherein the transition metal is Fe^{II}, Co^{II}, Ni^{II}, and Cu^{II}.

22. The method according to claim 19, wherein the contrast agent is water-soluble.

23. The method according to claim 19, wherein A₁, A₂, and A₃ are the same.

24. The method according to claim 19, wherein the diagnostic method is medical imaging.

25. The method according to claim 19, wherein A1, A2, and A3 are independently selected from the group consisting of:

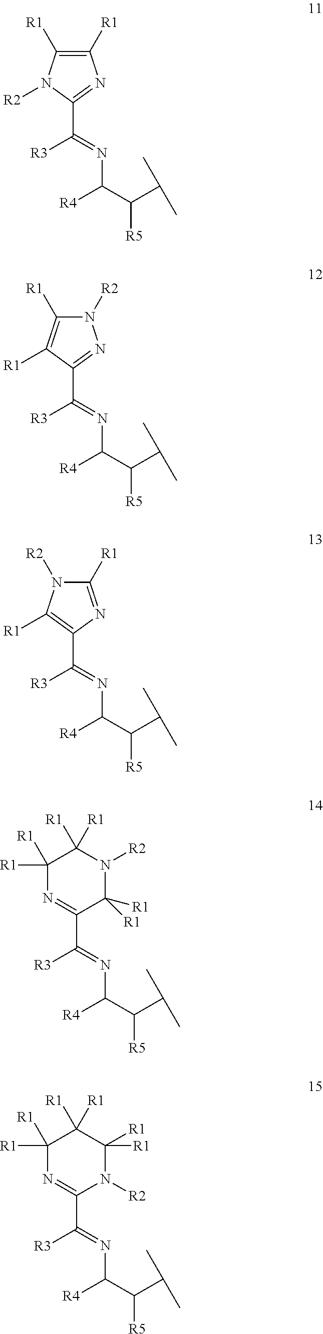

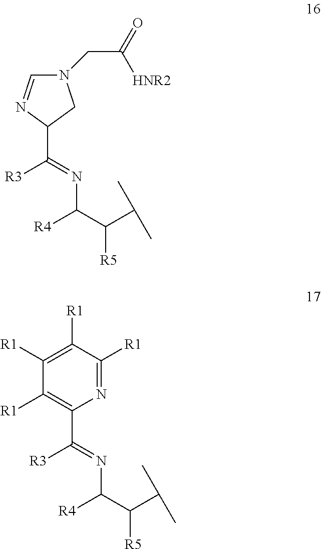

26. The method according to claim 19, wherein the counter ion(s) is/are selected from the group consisting of acetate (OAc⁻), chloride (Cl⁻), iodide (I⁻), bromide (Br⁻), nitrate (NO₃⁻), triflate (OTf⁻) and sulfate (SO₄²⁻).

27. The method according to claim 19, wherein
R₁, R₄, R₅, and R₆ are independently selected from the group consisting of: H, F, Cl, Br, I methyl, OMe, OH, and CF₃, wherein R₂ is H, or OH; and
R₃═H or CH₃.

28. The method according to claim 19, wherein
R₁, R₂, R₃, R₄, R₅, and R₆ are independently selected from the group consisting of:
H, OH, SH, CF₃, CN, halogen, optionally substituted C₁₋₄ alkyl, C₁₋₄ heteroalkyl, C₃₋₇ cycloalkyl, C₃₋₇ heterocycloalkyl, C₄₋₁₂ aryl C(O)NH₂ or C(O)OH and C₄₋₁₂ heteroaryl groups.
wherein R₆ is H, OH, or an C₁₋₄ alkyl; and
wherein if R² does not equal H, OH, SH, C(O)NH₂, or C(O)OH, at least one of R₁, R₃, R₄, and R₅ must equal OH, SH, NH₂, C(O)NH₂, C(O)OH.

29. The method according to claim 19, wherein
R₁═H, F, Cl, Br, I, CH₃, OCH₃, OH or CF₃;
R₂, R₄, R₅, and R₆═H; and
R₃═H or CH₃.

30. The method according to claim 19, wherein
at least one of R₁═OH;
R₂, R₄, R₅, and R₆═H; and
R₃═H or CH₃.

31. The method according to claim 19, wherein one or more of R₂, R₃, R₄, R₅, R₆ and each of R₁ is coupled to a probe, or label, wherein the probe or label can be an antibody, peptide, or a dye, or ¹⁹F-based probe.

32. The method according to claim 19, wherein A₁, A₂, and A₃ are selected from

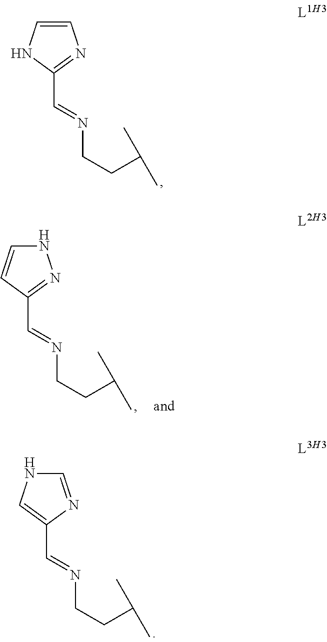

33. The method according to claim 32, wherein the counter ion is trifluoromethanesulfonate or chloride and the metal is Fe or Co or Ni.

34. A contrast agent of the formula [N(A₁,A₂,A₃) M](counter ion(s)) for use in a diagnostic method practiced on the human or animal body, wherein
N is a nitrogen atom;
M is a divalent metal ion selected from transition metals of the group: V^{II}, Cr^{II}, Fe^{II}, Co^{II}, Ni^{II}, and Cu^{II};
the counter ion(s) being pharmaceutically acceptable; and wherein
A₁, A₂, and A₃ are independently selected from the group of ligands consisting of: 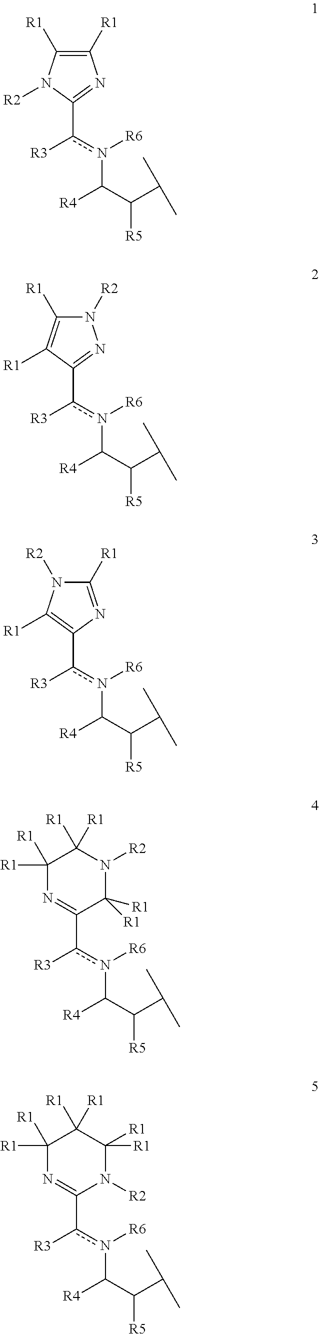 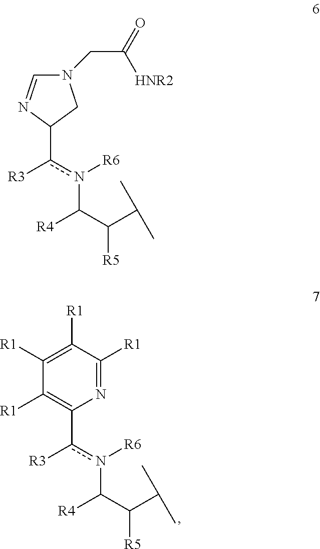
wherein [symbol] denotes a single or a double bond, wherein R₆ is absent if there is a double bond; and
R₂, R₃, R₄, R₅, R₆, and each of R₁, are independently selected from the group consisting of: H, OH, SH, CF₃, CN, C(O)NH₂, C(O)H, C(O)OH, halogen, optionally substituted C₁₋₄ alkyl; C₁₋₄ heteroalkyl, C₃₋₇ cycloalkyl, C₃₋₇ heterocycloalkyl, C₄₋₁₂ aryl and C₄₋₁₂ heteroaryl groups, R^{k}, SR^{k}, S(O)R^{k}, S(O)₂R^{k}, S(O)OR^{k}, S(O)₂OR^{k}, OS(O)R^{k}, OS(O)₂R^{k}, OS(O)OR^{k}, OS(O)₂R^{k}, OR^{k}, P(O)(OR^{k})(OR^{L}), OP(O)(OR^{k})(OR^{L}), SiR^{k}R^{L}R^{m}, C(O)R^{k}, C(O)OR^{k}, C(O)N(R^{L})R^{k}, OC(O)R^{k}, OC(O)OR^{k}, OC(O)N(R^{k})R^{L},
wherein R, R^{k}, R^{L}, and R^{m} are independently selected from the group consisting of H and optionally substituted C₁₋₄ alkyl; C₁₋₄ heteroalkyl, C₃₋₇ cycloalkyl, C₃₋₇ heterocycloalkyl, C₄₋₁₂ aryl, or C₄₋₁₂ heteroaryl groups, wherein two or more of R^{k}, R^{L} and R^{m} may form, together with each other, one or more optionally substituted aliphatic or aromatic carbon cycles or heterocycles; and wherein one or more of R₂, R₃, R₄, R₅, R₆ and each of R₁, can be coupled to a probe, or label; and
wherein for ligands 1-6 the following conditions additionally apply:
if R₆ is one of H, OH, NH₂, NHR, SH, C(O)NH₂, C(O)NHR, or C(O)OH, then R₁-R₅ can be independently selected from the group as indicated above;
if R₆ is absent or not one of H, OH, NH₂, NHR, SH, C(O)NH₂, C(O)NHR, or C(O)OH, then R₂ must be H, OH, NH₂, NHR, SH, C(O)NH₂, C(O)NHR, or C(O)OH and/or at least one of R₁, R₃, R₄ and R₅ must be OH, NH₂, NHR, SH, C(O)NH₂, C(O)NHR, or C(O)OH; and
wherein for ligand 7 the following conditions additionally apply:
if R₆ is one of H, OH, NH₂, NHR, SH, C(O)NH₂, C(O)NHR, or C(O)OH, then R₁, R₃, R₄ and R₅ can be independently selected from the group as indicated above.
if R₆ is absent or R₆ is not one of H, OH, NH₂, NHR, SH, C(O)NH₂, C(O)NHR, or C(O)OH then at least one of R₁, R₃, R₄ and R₅ must be OH, NH₂, NHR, SH, C(O)NH₂, C(O)NHR, or C(O)OH.

35. A pharmaceutical composition comprising a contrast agent as defined in claim 34 and at least one pharmaceutically acceptable excipient.

36. A method of in vitro medical imaging, especially of diagnostic imaging, comprising administering a compound as defined in claim 34 to a sample.

37. A contrast agent as defined in claim 34, wherein the pharmaceutically acceptable counterion(s) is/are selected from the group consisting of acetate (OAc⁻), chloride (Cl⁻), iodide (I⁻), bromide (Br⁻), nitrate (NO₃⁻), triflate (OTf) and sulfate (SO₄²⁻).
